SEQUENCE LISTING (10 sequences)

SEQ ID NO: 1 - DNA, 21 bp, Artificial Sequence
  Synthetic oligonucleotide
atcctggagtcctgggatcga

SEQ ID NO: 2 - DNA, 21 bp, Artificial Sequence
  Synthetic oligonucleotide
agagggagaagcaggctccat

SEQ ID NO: 3 - DNA, 14 bp, Artificial Sequence
  Synthetic oligonucleotide
cccacatcgggctc

SEQ ID NO: 4 - DNA, 22 bp, Artificial Sequence
  Synthetic oligonucleotide
ggaaggagcctcggtgtccawc

SEQ ID NO: 5 - DNA, 24 bp, Artificial Sequence
  Synthetic oligonucleotide
ccacccctagttcgtttcccagag

SEQ ID NO: 6 - DNA, 20 bp, Artificial Sequence
  Synthetic oligonucleotide
gggcgaccttatcgctctct

SEQ ID NO: 7 - DNA, 18 bp, Artificial Sequence
  Synthetic oligonucleotide
ttccccctcgtcctgtca

SEQ ID NO: 8 - DNA, 21 bp, Artificial Sequence
  Synthetic oligonucleotide
gcctgttccagtgyctaacaa

SEQ ID NO: 9 - DNA, 27 bp, Artificial Sequence
  Synthetic oligonucleotide
cactaacagtccccactaaaccatatc

SEQ ID NO: 10 - DNA, 22 bp, Artificial Sequence
  Synthetic oligonucleotide
ttggcctgttctcccacgtgcc

(19) United States
(12) Patent Application Publication
(10) Pub. No.: US 2014/0315277 A1
(43) Pub. Date: Oct. 23, 2014

(54) NOVEL METHOD

(71) Applicants: Bruno Rene ANDRE, Rixensart (BE); Benoit Paul Suzanne Champluvier, Rixensart (BE); Benedicte Van Der Hayden, Rixensart (BE)

(72) Inventors: Bruno Rene ANDRE, Rixensart (BE); Benoit Paul Suzanne Champluvier, Rixensart (BE); Benedicte Van Der Hayden, Rixensart (BE)

(73) Assignee: GlaxoSmithKline Biologicals s. a., Rixensart (BE)

(21) Appl. No.: 14/184,740

(22) Filed: Feb. 20, 2014

Related U.S. Application Data
(63) Continuation of application No. 13/127,825, filed on May 5, 2011, now abandoned.
(60) Provisional application No. 61/111,481, filed on Nov. 5, 2008.

(51) Int. Cl. C12N 7/00

(52) U.S. Cl. CPC C12N 7/00
USPC 435/238; 435/235.1

(57) ABSTRACT

The present invention relates to a method for degrading host cell nucleic acids associated with a virus or a viral antigen thereof produced by cell culture, the method comprising at least two steps of nucleic acids degradation with a compound selected from i) an endonuclease and ii) a DNA alkylating agent.

This application is a continuation of U.S. application Ser. No. 13/127,825 filed May 5, 2011 which is the U.S. National Stage of International Application No. PCT/EP2009/064537, filed 3 Nov. 2009, which claims benefit to U.S. Application No. 61/111,481 filed 5 Nov. 2008, each of which is incorporated herein by reference.

### STATEMENT REGARDING FEDERALLY SPONSORED RESEARCH

Aspects of this invention were made with United States government support pursuant to Contract # HHS0100200600011C, from the Department of Health and Human Services; the United States government may have certain rights in the invention.

### TECHNICAL FIELD

The present invention relates to a cell culture-based method of producing viruses or viral antigens, to viruses or viral antigens obtainable by this method and to vaccines containing such viruses or viral antigens. In particular, the invention provides a method for reducing the amount and/or the size of contaminating residual nucleic acids from host cells. According to the invention, host cell nucleic acids are degraded through the implementation of at least two distinct degradation steps performed by an endonuclease, such as BENZONASE™, and/or by a DNA alkylating agent, such as beta-propiolactone (BPL), or by a combination thereof.

### TECHNICAL BACKGROUND

The development of cell culture-based technologies as an alternative to the traditional egg-based production systems for the manufacture of viral vaccines likely appears as the most rapid and most promising solution to overcome drawbacks and constraints associated with egg-based traditional systems. Commercial productions of viral vaccines typically require large quantities of virus as an antigen source. However, the egg-based process is vulnerable due to the varying biological quality of eggs and it lacks flexibility because of the logistic problems due to non-availability of large quantities of suitable eggs.

Cell culture systems appear as a suitable alternate mode of vaccine preparation, simpler, flexible, and consistent, allowing to improve possibilities of up-scaling vaccine production capacities and thus to reach large quantities of virus, if needed. For example, in response to a natural pandemic threat or a to a terrorist attack.

Commercial quantities of virus may be achieved by replicating a seed virus in a cell culture system. Cell culture systems suitable for viral replication or for preparation of viral antigens include mammalian, avian or insect cells.

However, it is known in the art that a risk associated with using cell culture for vaccine production is the exposure of vaccine recipients to contaminating intact cells, cellular components and/or residual cellular DNA.

If unmodified from their naturally occurring states, cell cultures have a limited ability to reproduce, and subsequently, are impractical and inefficient for producing the amount of material necessary for a commercial vaccine. Consequently, for manufacturing purposes, it is preferred that the cells be modified to be “continuous” or “immortalized” cell lines to increase the number of times they can divide. Many of these modifications employ mechanisms similar to those which are implicated in oncogenic cells. As such, there is thus a concern that any residual material from the cell culture process, such as host cell DNA, be removed from the final formulation of a vaccine manufactured in these systems, to remove any potential oncogenic material from the final product.

Therefore, cell culture-based viruses or viral antigens for vaccine purposes have to be properly and carefully isolated from their cellular environment, while preserving their antigenic and immunogenic properties.

Given the stricter requirements imposed by regulatory authorities, there remains a need to not only provide viruses or viral antigens with a residual DNA content as low as possible, but also to provide a method allowing to monitor the profile, including the size, the distribution and the amount of the cellular DNA at each step of the production process. In particular, there is a need to develop vaccine doses comprising viruses or viral antigens containing less than 10 ng cellular residual DNA, which cellular residual DNA should not exceed 300 base pairs.

European patent 0870508 discloses the use a DNA digesting enzyme as a DNA degrading agent.

International application WO 2007/052163 discloses the use of beta-propiolactone (BPL), as a DNA degrading agent.

The method according to the present invention allows to achieve both a high DNA fragmentation and a low content of residual DNA, while achieving high virus yield and preserving virus or viral antigens immunogenicity.

### SUMMARY OF THE INVENTION

Accordingly, in a first aspect of the present invention, there is provided a method for degrading host cell nucleic acids associated with a virus or a viral antigen thereof produced by cell culture, the method comprising at least two steps of nucleic acids degradation with a compound selected from i) an endonuclease and ii) a DNA alkylating agent.

In a second aspect of the present invention, there is provided a method for producing a virus or a viral antigen thereof in cell culture comprising the steps of:
(a) providing a population of cells cultured in a cell culture medium,
(b) inoculating the population of cells with a virus,
(c) culturing the population of cells so as to allow the virus to replicate,
(d) collecting the produced virus thereby providing a viral harvest, and
(e) isolating the virus,
   the method comprising at least two steps of host cell nucleic acids degradation with a compound selected from i) an endonuclease and ii) a DNA alkylating agent.

In a third aspect, there is provided a composition comprising a virus, or a viral antigen thereof, obtainable by the method of the invention.

In a fourth aspect, there is provided a composition comprising a cell culture-produced virus, or viral antigen thereof, having a residual host cell DNA content of less than 10 ng, less than 5 ng, less than 1 ng, less than 100 pg or less than 10 pg as measured by THRESHOLD™ assay.

In a fifth aspect, there is provided a composition comprising a cell culture-produced virus, or viral antigen thereof, having a residual host cell DNA content whose size is less than 500 base pairs, less than 300 base pairs, less than 200 base pairs and less than 100 base pairs, as measured by Southern-blot.

In a further aspect, there is provided an immunogenic composition comprising a virus, or a viral antigen thereof, of the invention admixed with a suitable pharmaceutical carrier.

In a still further aspect, there is provided a method for monitoring residual host cell DNA during a cell culture-based method of producing a virus, including the method of the present invention, which method allows measuring both the amount and the size of the residual cellular DNA.

In a still further aspect, there is provided a method for purifying a virus or a viral antigen thereof produced in cell culture comprising at least the following steps:
(a) a sucrose gradient ultracentrifugation step, wherein the sucrose gradient comprises a detergent so as to spilt the virus, and
(b) incubate the split virus pool collected from the sucrose gradient in the presence of 0.1% to 0.5% of a non-ionic detergent for 1 to 5 days.

In a still further aspect, there is provided a method for purifying a virus or a viral antigen thereof produced in cell culture comprising at least one size exclusion chromatography step performed in the presence of a zwitterionic detergent.

In a still further aspect, there is provided a method for purifying a virus or a viral antigen thereof produced in cell culture comprising at least the following steps:
(a) one sucrose gradient ultracentrifugation step, and
(b) one chromatography step.

### DESCRIPTION OF DRAWINGS

FIG. 1: Validation of the amplification of SINE and LINE sequences by quantitative PCR (Q-PCR). FIG. 1A shows the curves established for SINE and LINE amplifications performed on MDCK DNA double digested with the restriction enzymes EcoRI/XhoI. FIG. 1B shows an agarose gel loaded with aliquots originating from the Q-PCR reactions performed to amplify the SINE and LINE sequences. Ct is for THRESHOLD™ cycle.

FIG. 2: Diagram showing the primers used for amplifying the sequences of 99 base pairs (bp), 185 base pairs (bp) and 280 base pairs (bp) of EB66™ cells by quantitative PCR (Q-PCR).

FIG. 3: Profile of residual host cell DNA present in a cell culture-produced Influenza virus harvest obtained by collecting the cell culture medium after MDCK cells were inoculated and infected with the virus. Comparison of the DNA profiles—no DNA degradation step versus one DNA degradation step implemented during the culture phase. FIG. 3 shows a picture taken under UV light of an agarose gel loaded with DNA samples prepared from the indicated virus harvest, JP115, NCP117, JP125 and NCP127. D is for DNase and R is for RNase.

FIG. 4: Characterization of residual MDCK DNA in the purified bulk of cell culture-produced Influenza viruses subjected to two DNA degradation steps performed with an endonuclease and with a DNA alkylating agent. FIG. 4A shows an autoradiography obtained after analysing the DNA content of the purified bulks of samples JP115 (lane 6) and JP125 (lane 8) by Southern-blot, as indicated by the arrows. Lanes 10 to 15 have been loaded with the indicated known amounts of MDCK control DNA digested with the restriction enzyme Sau3, on which semi-quantification of JP115 and JP125 DNA was based. FIG. 4B shows an autoradiography obtained after analysing the DNA content of the purified bulk of sample NCP124 (lane 5, as indicated by an arrow) by Southern-blot. Lanes 7 to 12 have been loaded with the indicated known amounts of MDCK control DNA digested with the restriction enzyme Sau3, on which semi-quantification of NCP124 DNA was based

FIG. 5: Immunogenicity of Influenza virus antigen produced on MDCK cells and subjected to multiple DNA degradation steps performed with an endonuclease and with a DNA alkylating agent in a naive mouse model. FIG. 5A shows the HI antibody response induced by the formulation derived from the sample NCP124. FIG. 5B shows the neutralizing antibodies response induced by the formulation derived from the sample NCP124.

FIG. 6: Immunogenicity of Influenza virus antigen produced on MDCK cells and subjected to multiple DNA degradation steps performed with an endonuclease and with a DNA alkylating agent in a primed mouse model. FIG. 6A shows the HI antibody response induced by the formulation derived from the sample NCP124. FIG. 6B shows the neutralizing antibodies response induced by the formulation derived from the sample NCP124.

### DETAILED DESCRIPTION

The invention provides a cell culture-based method of producing viruses or viral antigens. This method is intended for limiting both the size and the amount of residual host cell nucleic acids, DNA in particular, remaining associated with the purified viruses or viral antigens. In particular, the invention provides an improved method of DNA degradation and a better elimination of degraded DNA. According to the invention, at least two host cell nucleic acids degradation steps are implemented in the method, using a endonuclease, such as BENZONASE™, and/or a DNA alkylating agent, such as BPL (beta-propiolactone), or a combination thereof. This method may be used to treat a range of cell-culture produced viruses or viral antigens.

The method of the invention is amenable to a wide range of viruses, any virus which is the target of a vaccine, including, but not limited to, adenoviruses, hepadnaviruses, herpes viruses, orthomyxoviruses, papovaviruses, paramyxoviruses, picornaviruses, poxviruses, reoviruses and retroviruses. In particular, the method of invention is suitable for enveloped viruses, such as myxoviruses. In one embodiment, the viruses produced by the method of the invention belong to the family of orthomyxoviruses, in particular, influenza virus.

Viruses or viral antigens may be derived from an Orthomyxovirus, such as influenza virus. Orthomyxovirus antigens may be selected from one or more of the viral proteins, including hemagglutinin (HA), neuraminidase (NA), nucleoprotein (NP), matrix protein (M1), membrane protein (M2), one or more of the transcriptase (PB1, PB2 and PA). Particularly suitable antigens include HA and NA, the two surface glycoproteins which determine the antigenic specificity of the Influenza subtypes.

Influenza virus is selected from the group consisting of human influenza virus, avian influenza virus, equine influenza virus, porcine (e.g. swine) influenza virus, feline influenza virus. Influenza virus is more particularly selected from strains A, B and C, preferably from strains A and B.

Influenza virus or antigens thereof may be derived from interpandemic (annual or seasonal) influenza strains. Alternatively, influenza virus or antigens thereof may be derived from strains with the potential to cause a pandemic outbreak (i.e., influenza strains with new hemaggultinin compared to hemagglutinin in currently circulating strains, or influenza strains which are pathogenic in avian subjects and have the potential to be transmitted horizontally in the human population, or influenza strains which are pathogenic to humans). Depending on the particular season and on the nature of the antigen included in the vaccine, the influenza virus or antigens thereof may be derived from one or more of the following hemagglutinin subtypes: H1, H2, H3, H4, H5, H6, H7, H8, H9, H10, H11, H12, H13, H14, H15 or H16. Preferably, the influenza virus or antigens thereof are from H1, H2, H3, H5, H7 or H9 subtypes.

The cells which are used in the method according to the invention can in principle be any desired cell type of cells which can be cultured in cell culture and which can support virus replication. They can be both adherently growing cells or cells growing in suspension. They can be either primary cells or continuous cell lines. Genetically stable cell lines are preferred.

Mammalian cells are particularly suitable, for example, human, hamster, cattle, monkey or dog cells.

A number of mammalian cell lines are known in the art and include PER.C6, HEK cells, human embryonic kidney cells (293 cells), HeLa cells, CHO cells, Vero cells, and MDCK cells.

Suitable monkey cells are, for example, African green monkey cells, such as kidney cells as in Vero cell line. Suitable dog cells are, for example, kidney cells as in MDCK cell line.

Suitable mammalian cell lines for growing influenza virus include MDCK cells, Vero cells, or PER.C6 cells. These cell lines are all widely available, for instance, from the American Type Cell Culture (ATCC) collection.

According to a specific embodiment, the method of the invention uses MDCK cells. The original MDCK cell line is available from the ATCC as CCL-34, but derivatives of this cell line may also be used, such as the MDCK cells adapted to growth in suspension (WO 1997/37000).

Alternatively, cell lines for use in the invention may be derived from avian sources, such as chicken, duck, goose, quail or pheasant. Avian cell lines may be derived from a variety of developmental stages including embryonic, chick and adult. In particular, cell lines may be derived from the embryonic cells, such as embryonic fibroblasts, germ cells, or individual organs, including neuronal, brain, retina, kidney, liver, heart, muscle, or extraembryonic tissues and membranes protecting the embryo. Chicken embryo fibroblasts (CEF) may be used. Examples of avian cell lines include avian embryonic stem cells (WO01/85938) and duck retina cells (WO05/042728). In particular, the EB66™ cell line derived from duck embryonic stem cells is contemplated in the present invention (WO 2008/129058). Other suitable avian embryonic stem cells include the EBx cell line derived from chicken embryonic stem cells, EB45, EB14 and EB14-074 (WO2006/108846). This EBx cell line presents the advantage of being a genetically stable cell line whose establishment has been produced naturally and did not require any genetic, chemical or viral modification. These avian cells are particularly suitable for growing influenza viruses.

According to a particular embodiment, the method of the invention uses EB66™ cells.

Cell culture conditions (temperature, cell density, pH value, etc. . . . ) are variable over a very wide range owing to the suitability of the cells employed and can be adapted to the requirements of particular virus growth conditions details. It is within the skilled person's capabilities to determine the appropriate culture conditions, as cell culture is extensively documented in the art (see, for example, Tissue Culture, Academic Press, Kruse and Paterson, editors (1973), and R.I. Freshney, Culture of animal cells: A manual of basic technique, fourth edition (Wiley-Liss Inc., 2000, ISBN 0-471-34889-9).

In a specific embodiment, host cells used in the method described in the present invention are cultured in serum-free and/or protein-free media. A “serum-free medium” (SFM) means a cell culture medium ready to use that does not require serum addition allowing cell survival and cell growth. This medium may not necessarily be chemically defined and may contain hydrolyzates of various origin, from plant for instance. Such serum-free medium present the advantage that contamination with viruses, mycoplasma or unknown infectious agents can be ruled out. “Protein-free” is understood to mean cultures in which multiplication of the cells occurs with exclusion of proteins, growth factors, other protein additives and non-serum proteins, but can optionally include proteins such as trypsin or other proteases that may be necessary for viral growth. The cells growing in such culture naturally contain protein themselves.

Serum-free media are commercially available from numerous sources, for instance, VP SFM (Invitrogen Ref 11681-020), Opti-Pro (Invitrogen, Ref 12309-019), or EX-CELL (JHR Bioscience).

Cells may be grown in various ways, for instance, in suspension, or adhering to surfaces, including growth on microcarriers, or combinations thereof. Culturing can be done in dishes, flasks, roller bottles, or in bioreactors, using batch, fed-batch, semi-continuous or continuous systems, such as perfusion systems. Typically, cells are scaled-up from a master or working cell bank vial through various sizes of flasks or roller bottles and finally to bioreactors. In one embodiment, the cells employed according to the method of the invention are cultured on microcarrier beads in a serum-free medium in a stirred-bioreactor and the culture medium is provided by perfusion.

In an alternative embodiment, cells are cultured in suspension in a batch mode.

Prior to infection with the virus, cells are cultured around 37° C., more suitably at 36.5° C., at a pH ranging from 6.7 to 7.8, suitably around 6.8 to 7.5, and more suitably around 7.2

According to the method of the invention, the production of cell culture-based viruses includes generally the steps of inoculating the cultured cells with the viral strain to be produced and cultivating the infected cells for a desired period of time so as to allow virus replication.

In order to produce large quantities of cell-produced viruses, it is preferred to inoculate cells with the desired virus strain once cells have reached a high density. Usually, the inoculation is performed when the cell density is at least around 1.5×10⁶ cells/ml, suitably, around 3×10⁶ cells/ml, more suitably, around 5×10⁶ cells/ml, even more suitably 7×10⁶ cells/ml, or even higher. The optimal cell density for obtaining the highest virus production may vary according to the cell type used for the virus propagation.

The inoculation is carried out at an MOI (Multiplicity Of Infection) of about 10⁻¹ to 10⁻⁷, suitably about 10⁻² to 10⁻⁶, and more suitably, about 10⁻⁵.

The temperature and pH conditions for virus infection may vary. Temperature may range from 32° C. to 39° C. depending on the virus type. For Influenza virus production, cell culture infection may vary depending on the strain which is produced. Influenza virus infection is suitably performed at a temperature ranging from 32° C. to 35° C., suitably at 33° C. In one embodiment, the virus infection occurs at 33° C. In an alternative embodiment, the virus infection takes place at 35° C. Proteases, typically trypsin, may be added to the cell culture depending on the virus strain, to allow viral replication. The protease can be added at any suitable stage during the culture. Tryspin is preferably of non-animal origin, that is to say the protease is not purified from an animal source. It is suitably recombinantly produced in a micro-organism, such as bacterial, yeast or plant. Suitable examples of recombinant trypsin are Trypzean, a recombinant trypsin produced in corn (Prodigen, 101 Gateway Blvd, Suite 100 College Station, Tex. 77845. Manufacturer code: TRY), or TrpLE (Invitrogen) which is a trypsin-like enzyme expressed in fungus (WO2004/020612).

Once infected, cells may release into the culture medium newly formed virus particles, due to spontaneous lysis of host cells, also called passive lysis. Therefore, in one embodiment, cell-based viral harvest may be provided any time after virus inoculation by collecting the cell culture medium or supernatant. In a particular embodiment, the cell culture medium is collected by perfusion. This mode of harvesting is particularly suitable when it is desired to harvest cell-derived virus at different time points after virus inoculation, and pooling the different harvests, if needed.

Alternatively, after virus infection, cell-based virus may be harvested by employing external factor to lyse host cells, also called active lysis. However, contrary to the previous one, such a harvesting mode requires that the cell-based viral harvest be collected at a single time point, as actively lysing the cells will immediately terminate the cell culture.

Methods that can be used for active cell lysis are known. Useful methods in this respect are for example, freeze-thaw, solid shear, hypertonic and/or hypotonic lysis, liquid shear, high pressure extrusion, detergent lysis, or any combination thereof.

According to one embodiment, cell-based viral harvest may be provided any time after virus inoculation by collecting the cell culture medium or supernatant, lysing the inoculated cells or both.

Before harvesting, cell infection may last for 2 to 10 days. According to a specific embodiment, culture supernatants from days 3, 4 and 5 post-inoculation are harvested and pooled for further downstream processing (virus isolation). According to a distinct embodiment, cell culture supernatant is collected from day 5 post-inoculation. The optimal time to harvest the cell-produced virus is usually based on the determination of the infection peak. For example, the CPE (CytoPathic Effect) is measured by monitoring the morphological changes occurring in host cells after virus inoculation, including cell rounding, disorientation, swelling or shrinking, death, detachment from the surface. The detection of a specific viral antigen may also be monitored by standard techniques of protein detection, such as a Western-blot analysis. Harvest can then be collected when the desired detection level is achieved. In the particular case of influenza virus, the content of HA may be monitored any time post-inoculation of the cells with the virus, by the SRD assay (Wood, J M, et al. (1977). J. Biol. Standard. 5, 237-247), which is a technique familiar to a person skilled in the art. Additionally, the SRD assay may also be used for determining the optimal cell density range required to obtain an optimized virus yield.

The present invention employs an at least 2 steps treatment for degrading host cell nucleic acids, when producing a cell cultured-based virus or viral antigens. The nucleic acids degrading compound may be an endonuclease, a DNA alkylating agent or both. For example, a first treatment may be performed by an endonuclease, followed by a second treatment performed by a DNA alkylating agent and vice-versa. Accordingly, in one embodiment at least one endonuclease step and at least one DNA alkylation step are performed sequentially, in any order. Alternatively, the method according to the invention may implement two distinct steps of nucleic acids degradation, each performed by an endonuclease or each performed by a DNA alkylating agent. The first step of nucleic acids degradation and the second one may be performed at any time of the method according to the present invention. They may be implemented in a consecutive way, i.e. one step immediately after the previous one. Alternatively, the two steps of nucleic acids degradation may be separated by other steps occurring during the method according to the invention.

The invention is not limited to two steps of host cell nucleic acids degradation and also contemplates, in another embodiment, more than two steps of nucleic acids degradation, each additional step being performed by an endonuclease or a DNA alkylating agent and implemented in any order at any time.

According to one embodiment, at least one step of host cell nucleic acids degradation is implemented before harvesting the virus produced, i.e. during the cell culture phase, as opposed to the virus isolation phase. In the context of the present invention, the cell culture phase and the virus isolation phase are separated by the virus harvesting step. The cell culture phase is to be understood as encompassing any step preceding the virus harvesting step, while the virus isolation phase is to be understood as encompassing any step following said harvesting step. The cell culture phase comprises, in particular, the steps of (a) providing a population of cells in culture, (b) inoculating the population of cells with a virus, and (c) culturing the population of cells so as to allow the virus to replicate. The virus isolation phase is to be understood as any step aimed at purifying the virus comprised in the viral harvest, i.e. aimed at eliminating the host cell contaminants, including host cell nucleic acids.

In one embodiment, the at least two steps of nucleic acids degradation are performed with an endonuclease. In a particular embodiment, the endonuclease is added both during the cell culture phase and during the virus isolation phase. In a distinct embodiment, the endonuclease is added both to the viral harvest obtained after collecting the cell culture supernatant of infected cells and during the virus isolation phase.

In an alternative embodiment, at least one step of nucleic acids degradation is performed with an endonuclease and at least one step of nucleic acids degradation is performed with an alkylating agent. In a particular embodiment, the endonuclease is added during the cell culture phase and the DNA alkylating agent during the virus isolation phase. In a distinct embodiment, both the endonuclease and the DNA alkylating agent are added during the virus isolation phase.

When implemented during the cell culture phase, the host cell nucleic acids degradation is suitably performed by adding an endoncuclease to the cell culture. The endoncuclease may be added at any suitable step of the cell culture phase, including simultaneously to the inoculation of the cells with the virus or any appropriate time after the virus inoculation. Such an early addition, in the cell culture phase, allows to target nucleic acids degradation as soon as they are released from the cells into the culture medium, before the possible occurrence of complexes or aggregates, with cell debris in particular, which would interfere with the recognition of nucleic acids by the endonuclease. Another consequence of adding the endonuclease to the cell culture is that the endonuclease action can take place in the exact same conditions and at the same time as the normally programmed cell culture steps, like the virus inoculation step, and therefore, does not require to set a specific temperature incubation, nor an additional time period of incubation, which may otherwise affect the stability of the final product and slow down the process. The mode of endonuclease addition may vary, depending on the mode of culture employed. When cells are cultured in a bioreactor, the endonuclease may be added directly in the bioreactor containing the cell culture. Alternatively, when a perfusion system is used for providing the cell culture medium, the endonuclease may be added to the perfusion medium.

In one embodiment, implementing at least one DNA degradation step during the cell culture phase according to the method of the invention allows reducing the amount of DNA fragments 300 base pairs long by more than 50%, particularly, more than 60%, more particularly, more than 70% and even more than 80% before proceeding to the virus isolation phase, as measured by quantitative PCR (Q-PCR) and as compared with no DNA degradation implemented at all.

In a specific embodiment, the method of the present invention provides a viral harvest obtained by collecting the cell culture supernatant of infected cells, in which the amount of DNA fragments 300 base pairs long has been reduced by more than 50%, particularly, by more than 60%, more particularly, by more than 70% and even by more than 80%, as measured by quantitative PCR (Q-PCR) and as compared with a viral harvest obtained while no DNA degradation step was implemented.

In another embodiment, implementing at least one DNA degradation step during the cell culture phase according to the method of the invention allows reducing the amount of DNA fragments 60 base pairs long by more than 30%, particularly, more than 40%, more particularly, more than 50% and even more than 55%, before proceeding to the virus isolation phase, as measured by quantitative PCR (Q-PCR) and as compared with no DNA degradation implemented at all.

In a specific embodiment, the method of the present invention provides a viral harvest obtained by collecting the cell culture supernatant of infected cells, in which the amount of DNA fragments 30 base pairs long has been reduced by more than 40%, particularly, by more than 50%, more particularly, by more than 55%, as measured by quantitative PCR (Q-PCR) and as compared with a viral harvest obtained while no DNA degradation step was implemented.

According to a second embodiment, at least one step of host cell nucleic acids degradation is implemented in the virus harvest collected after cell infection. In particular, an endonuclease may be added in the container containing the collected virus harvest. When the virus harvest is collected daily over a few days after virus inoculation, daily harvests are pooled. The endonuclease may then be added after the pooling.

According to a third embodiment, at least one step of host cell nucleic acids degradation is implemented during the virus isolation phase. This degradation step may take place at any suitable stage of the virus isolation phase.

Viruses or viral antigens produced according to the method of the present invention may be subjected to further purification, using standard techniques employed for virus purification, irrespective of their production mode. For instance, the virus isolation phase of cell culture-based viruses may include a number of different filtration, concentration and/or other separation steps such as ultrafiltration, ultracentrifugation (including density gradient ultracentrifugation), chromatography (such as ion exchange chromatography) and adsorption steps in a variety of combinations. Such a purification allows, in particular, to eliminate the degraded host cell nucleic acids, so as to obtain a final purified product substantially free of residual host cell nucleic acids. By “substantially” is meant that the final purified product, i.e. the final purified virus or viral antigen thereof comprises less than 10 ng of DNA, in particular, less than 5 ng of DNA, more particularly, less than 1 ng of DNA, even more particularly, less than 0.1 ng of DNA, suitably, less than 100 pg of DNA and more suitably, less than 10 pg of DNA.

In one embodiment, the method according to the invention allows reducing the total DNA content by an at least 20 000-fold factor, suitably, an at least 40 000-fold factor, and more suitably, by an at least 50 000-fold factor, as compared to the content of DNA initially present in the virus harvest obtained by collecting the cell culture supernatant of infected cells.

In a further embodiment, the method according to the invention allows reducing the total DNA content by more than 90%, particularly, more than 95%, more particularly, more than 99%, and even more 99.9%, as measured by THRESHOLD™ assay and as compared to the content of DNA initially present in the virus harvest obtained by collecting the cell culture supernatant of infected cells.

In one embodiment, during the virus isolation phase, the method of the invention comprises at least one step selected from viral harvest clarification, ultrafiltration/diafiltration, ultracentrifugation and chromatography, or any combination thereof. Depending on the purity level that is desired, the above steps may be combined in any way.

In a particular embodiment, during the virus isolation phase, the method of the invention comprises at least a viral harvest clarification step, an ultrafiltration/diafiltration step thereby providing a retentate and one or two ultracentrifugation steps.

After collecting the virus-containing cell culture supernatant of infected cells, the provided viral harvest may be clarified in order to separate the virus from the cellular material, such as intact floating cells or cell debris. Clarification may be done by a filtration step. Suitable filters may utilize cellulose filters, regenerated cellulose filters, cellulose fibers combined with inorganic filter aids, cellulose filter combined with inorganic filter aids and organic resins, or any combination thereof, and polymeric filters. Although not required, a multiple filtration process may be carried out, like a two- or three-stage process consisting, for instance, in sequentially and progressively removing impurities according to their size, using filters with appropriate nominal pore size, in particular, filters with decreasing nominal pore size, allowing to start removing large precipitates and cell debris. In addition, single stage operations employing a relatively tight filter or centrifugation may also be used for clarification. More generally, any clarification approach including, but not limited to, dead-end filtration, depth filtration, microfiltration, or centrifugation, which provide a filtrate of suitable clarity to not foul the membrane and/or resins in subsequent steps, will be acceptable to use in the clarification step of the present invention. In one embodiment, the viral clarification step is performed by depth filtration, in particular, using a three-stage train filtration composed, for example, of three different depth filters with nominal porosities of 5 μm-0.5 μm-0.2 μm. In another embodiment, the viral harvest is pre-clarified by centrifugation and then clarified by depth filtration, for instance, using a filtration train composed of two different filters with nominal porosities of 0.5 μm-0.2 μm.

According to the present invention, the virus suspension may be subjected to ultrafiltration (sometimes referred to as diafiltration when used for buffer exchange), for concentrating the virus and/or buffer exchange. This step is particularly advantageous when the virus to be purified is diluted, as is the case when pooling viral harvest collected by perfusion over a few days post-inoculation. The process used to concentrate the virus according to the method of the present invention can include any filtration process where the concentration of virus is increased by forcing diluent to be passed through a filter in such a manner that the diluent is removed from the virus suspension whereas the virus is unable to pass through the filter and thereby remains in concentrated form in the virus preparation.

Ultrafiltration may comprise diafiltration which is an ideal way for removal and exchange of salts, sugars, non-aqueous solvents, removal of material of low molecular weight, of rapid change of ionic and/or pH environments. Microsolutes are removed most efficiently by adding solvent to the solution being ultrafiltered at a rate equal to the ultrafiltration rate. This washes microspecies from the solution at a constant volume, isolating the retained virus. Diafiltration is particularly advantageous when a downstream step requires that a specific buffer be used in order to get an optimal reaction. For example, implementing a diafiltration step before degrading host cell nucleic acids with an endonuclease may allow performing the endonuclease reaction in a buffer specific and optimal for that endonuclease. Concentration and diafiltration may also be implemented at any suitable step of the purification process, when it is wanted to remove undesirable compounds, such as sucrose, after a sucrose gradient ultracentrifugation, or such as formaldehyde, after a step of virus inactivation with formaldehyde. The system is composed of three distinct process streams: the feed solution (comprising the virus), the permeate and the retentate. Depending on the application, filters with different pore sizes may be used. In the present invention, the retentate contains the virus and can be used for further purification steps, if desired. The membrane composition may be, but is not limited to, regenerated cellulose, polyethersulfone, polysulfone, or derivatives thereof. The membranes can be flat sheets (also called fait screens) or hollow fibers.

In one embodiment, the virus isolation phase of the method of the invention comprises at least one ultrafiltration/diafiltration step, suitably at least two ultrafiltration/diafiltration steps.

In one particular embodiment, at least one step of degradation of the host cell nucleic acids is performed by adding an endonuclease to the retentate obtained after ultrafiltration/diafiltration.

In a specific embodiment, an endonuclease is added both during the cell culture phase and to the retentate obtained after an ultrafiltration/diafiltration was implemented during the virus isolation phase.

The virus suspension obtained according to the method of the present invention may be further purified, by methods generally known to the person skilled in the art, such as density gradient centrifugation, for instance sucrose gradient ultracentrifugation, and/or chromatography. However, in one embodiment, the virus isolating step of the method of the invention does not comprise any chromatography step.

Hence, in certain embodiments, the virus isolation phase comprises at least one step of sucrose gradient ultracentrifugation, a technique commonly used for isolating viruses and known in the art.

In a particular embodiment, the virus isolating step of the method of the invention comprises at least two steps of sucrose gradient ultracentrifugation.

In a distinct embodiment, the virus isolation phase comprises at least two steps of ultracentrifugation, one of them being possibly a sucrose gradient ultracentrifugation step.

According to the method of the invention, it may be possible to combine a purification step, such as sucrose gradient ultracentrifugation, with a virus splitting step. In particular, a splitting agent may be added to the sucrose gradient. This embodiment is particularly suitable, when it is desired to minimize the total number of steps of the method of the invention, as it allows, within a single operation, to both purify and split the virus. Hence, in certain embodiments, when at least one sucrose gradient ultracentrifugation is implemented, the sucrose gradient additionally comprises a splitting agent.

Alternatively, the virus splitting step of the method of the present invention is performed in batch.

It is also possible to isolate viruses by chromatography, including ion exchange, anionic or cationic, chromatography, size exclusion, such as gel filtration or gel permeation, chromatography, hydrophobic interaction chromatography, hydroxyapatite or any combination thereof. As mentioned above, the chromatography steps may be implemented in combination with other purifications steps, such as density gradient ultracentrifugation. The person skilled in the art will be aware of theses processes, and can vary the exact way of employing these additional steps to optimize the method of the invention.

A gel filtration chromatography may be more suitably employed, in particular at the end of the virus purification phase, when it is desired to further improve the elimination of host cell proteins contaminating the virus or viral antigens produced according to the method of the present invention. The person skilled in the art is familiar with this technique and will know how to make vary the conditions depending on the virus produced and the cells used. He will know how to monitor and evaluate the host cell protein content, by using, for instance, any known in the art method of protein detection, such as Western blot analysis or THRESHOLD™ assay. If implementing the gel filtration chromatography in the presence of ionic detergents, a better separation of the host cell protein contaminants is achieved. The detergents may be either ionic or zwitterionic, empigen in particular. Empigen is more suitably used for its ability to allow an efficient separation of host cell impurities, while preserving integrity of the purified virus or viral antigens. Detergents may be selected from deoxycholate, sarcosine, Sodium Lauryl Sarcosinate, empigen or any combination thereof. The detergent may be added at different substeps. For instance, it may be added initially to the virus- or viral proteins-containing samples which need to be purified. The resulting mixture may then undergo the gel filtration, or the detergent may first be left incubated in the sample for a period of time. Detergent may also be present in the equilibration buffer. When employed at different substeps, detergents are not necessarily identical. For instance, the detergent used in the sample may be distinct from the one used in the equilibration buffer. Within the same substep, detergent may also be understood as a mixture of one or more of them. In one embodiment, there is provided a method for purifying a virus or a viral antigen thereof produced in cell culture comprising at least one size exclusion chromatography step, for instance, gel filtration, performed in the presence of a zwitterionic detergent.

In one embodiment, the virus isolation phase of the method of the invention comprises a gel filtration chromatography. In a further embodiment, the gel filtration is performed in the presence of a zwitterionic detergent, which may be present in the sample to be purified, in the equilibration buffer or both. Detergents may be used at a varying concentration. It is particularly used between 0.001% to 1%, more particularly, between 0.005% and 0.5%, still more particularly, at 0.1%.

In certain embodiments, the virus isolation phase comprises at least one step of chromatography selected from anion exchange chromatography, hydrophobic interaction chromatography, and a combination thereof.

In a specific embodiment, the virus isolation phase comprises at least the combination of one sucrose gradient ultracentrifigation with at least one chromatography step, in particular hydroxyapatite, or size exclusion. This combination may be used as a more simple advantageous alternative to the implementation of two sucrose gradient ultracentrifugation steps, as it allows to increase virus or viral antigen recovery while maintaining its quality. If a split virus is desired, it may occur either during the sucrose gradient ultracentrifugation, by adding a splitting agent to the sucrose gradient, as described above, or in a batch mode, as discussed below, in particular, after the ultracentrifugation.

Accordingly, the present invention also contemplates a method for purifying a virus or a viral antigen thereof produced in cell culture comprising at least one sucrose gradient ultracentrifugation step and one chromatography step, in particular, hydroxyapatite.

According to the invention, the at least one step of host cell nucleic acids degradation implemented during the virus isolation phase may be performed by adding an endonuclease and/or an alkylating agent to any suitable step occurring during the purification. For instance, an endonuclease may be added after the ultrafiltration step. It may also be added after the virus splitting step, when a detergent is used as the splitting agent, as the detergent presence is expected to help dissociate DNA from aggregates, the formation of which may prevent the enzyme to operate in optimal conditions.

At the end of the virus isolation phase, the virus preparation is suitably subjected to sterile filtration, as is common in processes for pharmaceutical grade materials, such as immunogenic compositions or vaccines, and known to the person skilled in the art. Such sterile filtration can for instance suitably be performed by filtering the preparation through a 0.22 μm filter. After sterile preparation, the virus or viral antigens are ready for clinical use.

The present invention further relates to viruses and to compositions comprising viruses or viral antigens obtainable by a method according to the invention and to their use in medicine. They can be formulated by any known method to give a vaccine for administration to humans or animals. Therefore, immunogenic compositions, such as vaccines, comprising viruses or viral antigens of this type are also contemplated by the present invention.

The present invention also provides compositions, for instance, immunogenic compositions, such as vaccine doses, comprising viruses or viral antigens, in particular, cell culture-produced virus or viral antigen thereof, wherein the content of residual host cell DNA is less than 10 ng, suitably, less than 5 ng, and more suitably less than 1 ng, in particular, less than 100 pg and more particularly, less than 10 pg, as measured by THRESHOLD™ assay.

The present invention also provides compositions, for instance, immunogenic compositions, such as vaccine doses, comprising viruses or viral antigens, in particular, cell culture-produced virus or viral antigen thereof, wherein any residual host cell DNA has a size of less than 500 base pairs, suitably, less than 300 base pairs, more suitably, less than 200 base pairs, and even less than 100 base pairs.

In certain embodiments, the viruses and the compositions of the invention display a content of DNA fragments whose size is 60 base pairs of less than 1 ng, particularly, less than 0.5 ng, more particularly, less than 0.1 ng, and even less than 0.01 ng, as measured by quantitative PCR (Q-PCR).

In further embodiments, the viruses and the compositions of the invention display a content of DNA fragments whose size is 300 base pairs of less than 1 ng, particularly, less than 0.5 ng, more particularly, less than 0.1 ng, and even less than 0.01 ng, as measured by quantitative PCR (Q-PCR).

In still further embodiments, the viruses and the compositions of the invention display a content of DNA fragments whose size is 300 base pairs of less than 1 ng, particularly, less than 0.5 ng, more particularly, less than 0.1 ng, and even less than 0.01 ng, and a content of DNA fragments whose size is 60 base pairs of less than 1 ng, particularly, less than 0.5 ng, more particularly, less than 0.1 ng, and even less than 0.01 ng, as measured by quantitative PCR (Q-PCR).

Exemplary endonucleases suitable for use in the method of the present invention include BENZONASE™, PULMOZYME™, DNase I or any other DNase and/or RNase commonly used in the art. In one embodiment, the endonuclease is BENZONASE™ which rapidly hydrolyzes nucleic acids by hydrolysing internal phosphodiester bonds between specific nucleotides, thereby fragmenting cellular DNA.

The concentration, the temperature and the incubation time in which the endonuclease is employed is dependent on the step at which it is used. It is within the ability of the skilled person to find the optimal conditions for using endonucleases. It is preferably employed within the range of 1-300 units/ml. As a non limiting example, when added to the cell culture, BENZONASE™ is used at a concentration varying between 1 and 10 units/ml, in particular between 1 and 3 units/ml, suitably, at a concentration of 1 unit/ml. According to the manufacturer, BENZONASE™ is effective over a temperature range of 0-42° C., although 37° C. has been determined as the optimal temperature for its nucleic acids degrading activity. Depending on the temperature, however, it is recommended to adapt the incubation time. In principle, longer incubation times are required at lower temperatures to achieve the same result (see BENZONASE™ brochure Merck KGaA). When added to the cell culture, BENZONASE™ reaction occurs at the temperature used for cell culturing. When implemented later in the virus production process, while the virus-containing suspension should be more concentrated, BENZONASE™ may be used at a higher concentration, for instance, in a range varying from 50 to 300 units/ml, suitably from 60 to 200 units/ml, in particular, 100 units/ml. As a non-limiting example, the incubation time and temperature for the BENZONASE™ reaction, at any step of the virus purification phase, may be 1 hour at 37° C.

Alkylating agents for use in the invention include substances that introduce an alkyl radical into a compound. In particular, the alkylating agent is a monoalkylating agent, such as BPL or ethylenimine. WO 2007/052163 discloses the use of BPL for degrading any residual functional cell culture DNA remaining associated to the final product. BPL reacts with various biological molecules, in particular, it modifies the nucleic bases of the viral genome and blocks its replication (Budowsky et al. 1993, Vaccine, 11(3): 343-348). It can be rapidly inactivated by heating at 37° C. for two hours since it is completely hydrolysed to non-toxic products beta-propionic acid and an isomer of lacate. It does not require a neutralizing agent to stop its reaction. An excess of BPL can be neutralized easily by addition of thiosulfate while maintaining viral integrity. The optimal conditions for BPL treatment will be determined by monitoring the effect of BPL, in different conditions, on nucleic acids degradation, DNA in particular. Methods for measuring nucleic acids degradation are described and discussed below. These methods rely on measuring DNA size. Accordingly, if willing to assess DNA degradation efficiency, it is possible, for instance, to measure the DNA size before proceeding to a degradation step, proceed to the degradation step, and measure the DNA size after the degradation step occurred. Comparing the size before and after a DNA degradation step and observing a decrease in DNA size after degradation will be indicative of the efficiency of that step.

Host cell nucleic acids degradation may be achieved with less than 1% BPL. Suitably, BPL is used at a concentration ranging from 0.01% to 01%, more suitably 0.5%. The alkylating agent is suitably added to a buffered solution and the pH solution is maintained between 5 and 10. More suitably, the pH of the solution is maintained between 6 and 9. Even more suitably, the pH of the solution is maintained between 7 and 8. The incubation time may vary. In particular, BPL is suitably incubated for an overnight period. BPL is active in a wide range of temperature. In one embodiment of the present invention, BPL is incubated at a temperature ranging from 2 to 8° C. In a distinct embodiment, BPL is incubated at room temperature.

In one embodiment, BPL is used in a citrate-containing phosphate buffer.

BPL presents the advantage of permitting to achieve two effects within a single operation, namely, to degrade the contaminating host cell nucleic acids and to inactivate the viral preparation. However, the method according to the present invention is not limited to a single use of BPL, but also contemplates, as described above, more than one BPL step. BPL can be added at any suitable step of the present method. It is preferably not added to the cell culture, but added to any step of the virus isolation phase. It is also within the scope of the present invention to use, additionally, inactivating agents different from BPL. In the method of the invention, a BPL step is suitably used in combination with one endonuclease step. In particular, the endonuclease is added during the cell culture phase, or to the viral harvest, while BPL is added during the virus isolation phase. For instance, BPL is added to the clarified viral harvest, i.e. after the viral harvest was subjected to a clarification step. Alternatively, a BPL step is suitably used in combination with two endonuclease steps. In particular, one nuclease step is implemented during the cell culture phase, the second one is implemented during the virus isolation phase, for instance, after the clarified viral harvest was concentrated by ultrafiltration/diafiltration, and BPL is added at any step of the virus isolation phase, in particular, immediately after the second endonuclease step. In one embodiment, BPL is added to the clarified harvest, the clarified harvest is concentrated by ultrafiltration and an endonuclease is added to the retentate removed from the ultrafiltration system.

The immunogenic compositions, in particular vaccines, of the present invention will generally be formulated in a sub-virion form, e.g. in the form of a split virus, where the lipid envelope has been dissolved or disrupted, or in the form of one or more purified viral proteins (subunit vaccine). As an alternative, the immunogenic compositions may include a whole virus, e.g. a live attenuated whole virus, or an inactivated whole virus.

Methods of splitting viruses, such as influenza viruses, are well known in the art (WO02/28422). Splitting of the virus is carried out by disrupting or fragmenting whole virus whether infectious (wild-type or attenuated) or non-infectious (inactivated) with a disrupting concentration of a splitting agent. Splitting agents generally include agents capable of breaking up and dissolving lipid membranes. Traditionally, split influenza virus was produced using a solvent/detergent treatment, such as tri-n-butyl phosphate, or diethylether in combination with TWEEN™ (known as “TWEEN™-ether” splitting) and this process is still used in some production facilities. Other splitting agents now employed include detergents or proteolytic enzymes or bile salts, for example sodium deoxycholate. Detergents that can be used as splitting agents include cationic detergents e.g. cetyl trimethyl ammonium bromide (CTAB), other ionic detergents, e.g. sodium lauryl sulphate (SLS), taurodeoxycholate, or non-ionic detergents such as TWEEN™ or Triton X-100, or combination of any two or more detergents.

In one embodiment, the splitting agent is deoxycholate. In another embodiment, the splitting agent is Triton X-100. In a further embodiment, the method according to the invention uses a combination of Triton X-100 and sodium lauryl sulfate as splitting agents.

The splitting process may be carried out as a batch, continuous or semi-continuous process. When implemented in batch, the split virus may require an additional step of purification to eliminate the detergent, such as a chromatography step. It is not necessary to implement a splitting step as such, as it is possible to perform the splitting simultaneously with a purification step. For instance, a detergent may be added to the sucrose gradient aimed at purifying viruses by ultracentrifugation, as described above.

The split virus pool collected from the sucrose gradient may contain a residual fraction of virus still infectious, as commonly detected, for example, by measuring the Tissue Culture Infectious dose (TCID₅₀/ml) in the pool, which represents the amount of a virus capable of infecting 50% of cells. A series of successive dilutions of the infectious samples to be tested are performed and part of each dilution is used for inoculating infectable cells. After incubating the cells for a few days, so that the virus, if infectious, can replicate, the presence of the virus may be detected by two reading methods known to the skilled person, the analysis of the cytopathic effect (CPE) in cells and/or the hemagglutination assay with chicken red blood cells performed on the culture supernatant. The viral titer is then calculated according to the Reed and Muench method (Reed, L. J. and Muench, H., 1938, The American Journal of Hygiene 27: 493-497).

If it is desired to further increase the elimination of virus infectivity, a storage step of the split virus in the presence of a high non-ionic detergent concentration may be, optionally, implemented after collecting the split virus pool from the sucrose gradient ultracentrifugation step. The detergent used in this storage step may be either identical, or distinct, to the one used in the sucrose gradient ultracentrifugation step. The concentration of detergent may vary from 0.1% to 1%, suitably from 0.2% to 0.5% and, more suitably, is 0.3%. The storage may occur at any temperature, particularly between 4° C. and 37° C., and more particularly at room temperature. The storage may last from 1 to 5 days, and, more particularly, is 3 days. In one embodiment, there is provided a method for purifying a virus or a viral antigen thereof produced in cell culture comprising a sucrose gradient ultracentrifugation step, wherein the sucrose gradient comprises a detergent so as to spilt the virus, and an incubation step of the split virus pool collected from the sucrose gradient in the presence of 0.1% to 0.5% of a non-ionic detergent for 1 to 5 days, in particular Triton X-100.

In another embodiment, the method for degrading host cell nucleic acids according to the present invention, wherein a detergent-containing sucrose gradient ultracentrifugation step is performed, further comprises a consecutive storage step of the virus pool collected from the sucrose gradient in the presence of 0.1% to 0.5%, more particularly, 0.3% of a non-ionic detergent, in particular Triton X-100.

For the safety of vaccines, it may be necessary to reduce infectivity of the virus suspension along different steps of the purification process. The infectivity of a virus is determined by its capacity to replicate on a cell line. Therefore, the method according to the present invention, optionally, includes at least one virus inactivation step. As described earlier, the inactivation may be performed by using BPL at any suitable step of the method. Alternatively, inactivation may be achieved with other inactivating agents known in the art, such as heat, formaldehyde or UV. In a specific embodiment, the method according to the invention further comprises inactivating the virus with one or more inactivating agents, selected from BPL, formaldehyde and UV. In one specific embodiment, the method according to the invention further comprises at least one BPL treatment step. In a specific embodiment, the method according to the invention further comprises at least one BPL treatment step and at least one formaldehyde treatment step. Formaldehyde and BPL may be used sequentially, in any order, for instance, formaldehyde is used after the BPL. The conditions of viral inactivation may vary and will be determined, in particular, by assessing the residual virus infectivity by measuring the Tissue Culture Infectious dose (TCID₅₀/ml).

Immunogenic compositions of the present invention, including vaccines, can optionally contain the additives customary for vaccines, in particular substances which increase the immune response elicited in a patient who receives the composition, i.e. so-called adjuvants.

In one embodiment, immunogenic compositions comprise a virus or a viral antigen thereof obtainable according to the present invention admixed with a suitable pharmaceutical carrier. In a specific embodiment, they further comprise an adjuvant.

Adjuvant compositions may comprise an oil in water emulsion which comprise a metabolisable oil and an emulsifying agent. In order for any oil in water composition to be suitable for human administration, the oil phase of the emulsion system has to comprise a metabolisable oil. The meaning of the term metabolisable oil is well known in the art. Metabolisable can be defined as ‘being capable of being transformed by metabolism’ (Dorland's Illustrated Medical Dictionary, W.B. Sanders Company, 25th edition (1974)). The oil may be any vegetable oil, fish oil, animal oil or synthetic oil, which is not toxic to the recipient and is capable of being transformed by metabolism. Nuts, seeds, and grains are common sources of vegetable oils. Synthetic oils are also part of this invention and can include commercially available oils such as NEOBEE™ and others.

A particularly suitable metabolisable oil is squalene. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast, and is a particularly preferred oil for use in this invention. Squalene is a metabolisable oil by virtue of the fact that it is an intermediate in the biosynthesis of cholesterol (Merck index, 10th Edition, entry no. 8619). In a further embodiment of the invention, the metabolisable oil is present in the immunogenic composition in an amount of 0.5% to 10% (v/v) of the total volume of the composition.

The oil-in-water emulsion further comprises an emulsifying agent. The emulsifying agent may suitably be polyoxyethylene sorbitan monooleate. Further, said emulsifying agent is suitably present in the vaccine or immunogenic composition 0.125 to 4% (v/v) of the total volume of the composition.

The oil-in-water emulsion of the present invention optionally comprises a tocol. Tocols are well known in the art and are described in EP0382271. Suitably may be a tocol is alpha-tocopherol or a derivative thereof such as alpha-tocopherol succinate (also known as vitamin E succinate). Said tocol is suitably present in the adjuvant composition in an amount 0.25% to 10% (v/v) of the total volume of the immunogenic composition.

The method of producing oil-in-water emulsions is well known to the person skilled in the art. Commonly, the method comprises mixing the oil phase (optionally comprising a tocol) with a surfactant such as a PBS/TWEEN80™ solution, followed by homogenisation using a homogenizer, it would be clear to a man skilled in the art that a method comprising passing the mixture twice through a syringe needle would be suitable for homogenising small volumes of liquid. Equally, the emulsification process in microfluidiser (M110S Microfluidics machine, maximum of 50 passes, for a period of 2 minutes at maximum pressure input of 6 bar (output pressure of about 850 bar)) could be adapted by the man skilled in the art to produce smaller or larger volumes of emulsion. The adaptation could be achieved by routine experimentation comprising the measurement of the resultant emulsion until a preparation was achieved with oil droplets of the required diameter.

In an oil-in-water emulsion, the oil and emulsifier are in an aqueous carrier. The aqueous carrier may be, for example, phosphate buffered saline.

In particular, the oil-in-water emulsion systems of the present invention have a small oil droplet size in the sub-micron range. Suitably the droplet sizes will be in the range 120 to 750 nm, more particularly sizes from 120 to 600 nm in diameter. Even more particularly, the oil-in water emulsion contains oil droplets of which at least 70% by intensity are less than 500 nm in diameter, more particular at least 80% by intensity are less than 300 nm in diameter, more particular at least 90% by intensity are in the range of 120 to 200 nm in diameter.

The oil droplet size, i.e. diameter, according to the present invention is given by intensity. There are several ways of determining the diameter of the oil droplet size by intensity. Intensity is measured by use of a sizing instrument, suitably by dynamic light scattering such as the Malvern Zetasizer 4000 or suitably the Malvern Zetasizer 3000HS. A detailed procedure is given in Example 11.2. A first possibility is to determine the z average diameter ZAD by dynamic light scattering (PCS-Photon correlation spectroscopy); this method additionally give the polydispersity index (PDI), and both the ZAD and PDI are calculated with the cumulants algorithm. These values do not require the knowledge of the particle refractive index. A second mean is to calculate the diameter of the oil droplet by determining the whole particle size distribution by another algorithm, either the Contin, or NNLS, or the automatic “Malvern” one (the default algorithm provided for by the sizing instrument). Most of the time, as the particle refractive index of a complex composition is unknown, only the intensity distribution is taken into consideration, and if necessary the intensity mean originating from this distribution.

The adjuvant compositions may further comprise a Toll like receptor (TLR) 4 agonist. By “TLR4 agonist” it is meant a component which is capable of causing a signalling response through a TLR4 signalling pathway, either as a direct ligand or indirectly through generation of endogenous or exogenous ligand (Sabroe et al, JI 2003 p 1630-5). The TLR 4 may be a lipid A derivative, particularly monophosphoryl lipid A or more particularly 3 Deacylated monophoshoryl lipid A (3 D-MPL).

3D-MPL is available under the trademark MPL™ by GlaxoSmithKline Biologicals North America and primarily promotes CD4+ T cell responses with an IFN-g (Th1) phenotype. It can be produced according to the methods disclosed in GB 2 220 211 A. Chemically it is a mixture of 3-deacylated monophosphoryl lipid A with 3, 4, 5 or 6 acylated chains. In particular, in the adjuvant compositions of the present invention small particle 3 D-MPL is used. Small particle 3 D-MPL has a particle size such that it may be sterile-filtered through a 0.22 μm filter. Such preparations are described in International Patent Application No. WO 94/21292. Synthetic derivatives of lipid A are known and thought to be TLR 4 agonists including, but not limited to:
OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026)
OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (W099/64301 and WO 00/0462)
OM 197 MP-Ac DP (3S-,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127)

Other TLR4 ligands which may be used are alkyl Glucosaminide phosphates (AGPs) such as those disclosed in WO9850399 or U.S. Pat. No. 6,303,347 (processes for preparation of AGPs are also disclosed), or pharmaceutically acceptable salts of AGPs as disclosed in U.S. Pat. No. 6,764,840. Some AGPs are TLR4 agonists, and some are TLR4 antagonists. Both are thought to be useful as adjuvants. In addition, further suitable TLR-4 agonists are disclosed in US2003/0153532 and US2205/0164988.

The invention is particularly suitable for preparing influenza virus immunogenic compositions, including vaccines. Various forms of influenza virus are currently available. They are generally based either on live virus or inactivated virus. Inactivated vaccines may be based on whole virions, spilt virions or purified surface antigens (including HA). Influenza antigens can also be presented in the form of virosomes (nucleic acid-free viral-like liposomal particles).

Virus inactivation methods and splitting methods have been described above and are applicable to influenza virus.

Influenza virus strains for use in vaccines change from season to season. In the current interpandemic period, vaccines typically include two influenza A strains and one influenza B strain. Trivalent vaccines are typical, but higher valence, such as quadrivalence, is also contemplated in the present invention. The invention may also use HA from pandemic strains (i.e. strains to which the vaccine recipient and the general human population are immunologically naïve), and influenza vaccines for pandemic strains may be monovalent or may be based on a normal trivalent vaccine supplemented by a pandemic strain.

Compositions of the invention may include antigen(s) from one or more influenza virus strains, including influenza A virus and/or influenza B virus. In particular, a trivalent vaccine including antigens from two influenza A virus strains and one influenza B virus strain is contemplated by the present invention.

The compositions of the invention are not restricted to monovalent compositions, i.e. including only one strain type, i.e. only seasonal strains or only pandemic strains. The invention also encompasses multivalent compositions comprising a combination of seasonal strains and/or of pandemic strains. In particular, a quadrivalent composition, which may be adjuvanted, comprising three seasonal strains and one pandemic strain falls within the scope of the invention. Other compositions falling within the scope of the invention are a trivalent composition comprising two A strains and one B strain, such as H1N1, H3N2 and B strains, and a quadrivalent composition comprising two A strains and two B strains of a different lineage, such as H1N1, H3N2, B/Victoria and B/Yamagata.

HA is the main immunogen in current inactivated influenza vaccines, and vaccine doses are standardized by reference to HA levels, typically measured by SRD. Existing vaccines typically contain about 15 μg of HA per strain, although lower doses can be used, e.g. for children, or in pandemic situations, or when using an adjuvant. Fractional doses such as a half (i.e. 7.5 μg HA per strain) or a quarter have been used, as have higher doses, in particular, 3× or 9× doses. Thus immunogenic compositions of the present invention may include between 0.1 and 150 μg of HA per influenza strain, particularly, between 0.1 and 50 μg, e.g. 0.1-20 μg, 0.1-15 μg, 0.1-10 μg, 0.1-7.5 μg, 0.5-5 μg, etc. Particular doses include about 15, about 10, about 7.5, about 5 μg per strain, about 3.8 pg per strain and about 1.9 μg per strain.

Once an influenza virus has been purified for a particular strain, it may be combined with viruses from other strains to make a trivalent vaccine, for example, as described above. It is more suitable to treat each strain separately and to mix monovalent bulks to give a final multivalent mixture, rather than to mix viruses and degrade DNA and purify it from a multivalent mixture.

The method of the present invention is particularly suitable as the nucleic acids degradation treatment employed in the method allows to preserve immunogenicity of the virus or viral antigen produced by the method. In particular, influenza virus, more particularly HA antigen, produced by the method of the invention is at least as immunogenic as the traditional egg-produced HA antigen. How to evaluate immunogenicity is part of the skilled in the art person's knowledge who is familiar with classical techniques for that purpose.

Measurement of residual host cell nucleic acids is within the normal capabilities of the person skilled in the art. The assay used to measure both DNA amount and DNA size will typically be a validated assay. The performance characteristics of a validated assay can be described in mathematical and quantifiable terms, and its possible sources of error will have been identified. The assay will generally have been tested for characteristics such as accuracy, precision, specificity. Once an assay has been calibrated, for instance against known standard quantities of host cell DNA, and tested, then the quantitative DNA measurements can be routinely performed. As examples, three principle techniques for DNA quantification can be used. The first method, the THRESHOLD™ System from Molecular Device is a sensitive and accurate method that allows to detect the presence of total DNA, irrespective of its size. Accordingly, in the sense of the present invention, the terms “total DNA” is to be understood as DNA of any size. This method however does not allow specifying the size of the residual DNA that is detected. Hybridization methods, such as Southern-blots, and quantitative PCR (Q-PCR) may be used in order to characterize the size of residual host cell DNA. All these techniques can be used for monitoring the amount and the size of residual host cell DNA along the different steps of the method of the invention. Southern-blots allow visualizing the size distribution of residual host cell DNA, in a direct way. When compared to a known amount of appropriate standard DNA, for instance, host cell genomic DNA, the size distribution can be semi-quantified, for instance by Phospholmager (Storm 860; Amersham Biosciences), using the ImageQuant TL analysis software (Amersham). In other words, a Southern-blot analysis allows determining what size, or sizes, is the DNA present within a sample to be tested and in what amount. Q-PCR, on the other hand, allows targeting any desired specific sequence, of any desired size, and to quantitate its presence in the sample to be tested. Because the method according to the present invention is intended for highly reducing the size and the amount of residual host cell DNA, due to the very low amount of residual DNA that is expected, sensitivity of the method used for measuring residual DNA may become an issue. In particular, large quantities of starting material may be required and the implementation of a DNA concentration step may be needed. A non-limiting way of overcoming this type of issue is to target by Q-PCR sequences of appropriate size which are highly abundant in the genome of the host cells which have been used for producing the virus. For instance, two highly repetitive sequences have been identified in the canine genome, whose respective sizes are 63 base pairs and 314 base pairs. Selecting these sequences increase the sensitivity of the assay, when the samples to be tested originate from MDCK cells which have been derived from dog kidneys. These two sequences are called the Short Interspersed repetitive DNA elements (SINE) and the Long Interspersed repetitive DNA elements (LINE) (Bentolila et al. 1999, Mamm. Genome 10(7):699-705). These SINE and LINE sequences have also been identified in other species, such as in duck (Walker, J. A., et al. 2004. Genomics. 83(3): 518-527) Therefore, it is within the scope of the invention to select any appropriate sequence of any desired species genome, design primers allowing to amplify it by Q-PCR and use it as an analytical tool for evaluating the size distribution and amount of residual DNA. By increasing the sensitivity of the assay, the quantity of starting material, and the DNA concentration in said material, are not so critical anymore. It may be possible, for example, to perform the Q-PCR directly from the virus preparation obtained at any step of the production process, in particular, the purified virus obtained at the end of the method according to the invention, without proceeding to any previous step of DNA extraction and/or concentration. As PCR inhibition may take place during the reaction, due to the presence of potential inhibitory molecules in the samples to be tested, it may be desirable to perform each Q-PCR reaction in duplicate. In one of the two reactions, a known amount of DNA originating from the same source as the one used for producing the virus should be spiked in order to estimate the Q-PCR inhibition, if any, and correct the final value, if needed. When a step of DNA extraction/concentration is implemented, due to the potential loss of material which may occur during that step, it may be useful to assess the efficiency of DNA recovery. For instance, before proceeding to the extraction/concentration of DNA, samples may be spiked with known amounts of specific external DNA amplicons of various size, as the extent of the loss may vary according to the size of the DNA fragments. In parallel of the Q-PCR intended for amplifying the target sequences, Q-PCR amplifying the different external DNA amplicons are performed to determine the DNA recovery of the fragments that were spiked. When the recovery is not total, the final value estimating the amount of the desired target sequence is corrected according to the recovery percentage, so as to accurately reflect the amount of DNA present in the sample, and not underestimate it. Q-PCR is an advantageous technique in that it is possible to quantitate any desired DNA sequence, of any desired length, and allowing to quantitate a number of different sequences within a single sample. When proceeding to the Q-PCR reaction, several primer sets may be combined within the same reaction tube, or the same sample may be aliquoted in a number of aliquots matching the number of primer sets, so that PCR conditions may vary according to the features of each primer set.

According to an aspect of the present invention, it is thus possible to monitor the DNA profile along the different steps of the method of cell culture-based virus production. In particular, the total DNA amount may be monitored, for instance, by measuring it by the THRESHOLD™ assay, allowing to establish a clearance factor for each step of the process and a total clearance factor reflecting the efficiency of each step and the overall efficiency of the whole process, respectively, in reducing total DNA amount. The amount of any specific sequence of any desired size may also be monitored along the virus production process by designing appropriate set of primers and amplifying them by Q-PCR. Also, the distribution of residual cellular DNA may be evaluated, at any time of the process, by Southern-blot analysis.

In certain embodiments, a virus or a viral antigen thereof obtained according to the method of the invention comprises less than 1 ng, particularly, less than 0.5 ng, more particularly less than 0.1 ng, and even less than 0.01 ng of DNA fragments whose length is 60 base pairs.

In further embodiments, a virus or viral antigen of the present invention comprises less than 0.5 ng, more particularly less than 0.1 ng, and even less than 0.01 ng of DNA fragments whose length is 300 base pairs.

In still further embodiments, a virus or viral antigen of the present invention comprises less than 1 ng, particularly, less than 0.5 ng, more particularly less than 0.1 ng, and even less than 0.01 ng of DNA fragments whose length is 60 base pairs and less than 0.5 ng, more particularly less than 0.1 ng, and even less than 0.01 ng of DNA fragments whose length is 300 base pairs.

The invention will be further described by reference to the following, non-limiting, examples.

### Example 1

### Production of Influenza Virus in MDCK Cells in the Presence of One Step of DNA Degradation Performed with an Endonuclease During the Virus Isolation Phase (JP115—Jiangsu B Strain, NCP117—New Caledonia A Strain)

### 1. Virus Multiplication

The MDCK adherent cells were grown on microcarriers in perfusion culture mode in a 20 liter stirred-bioreactor scale at 36.5° C. After the growth phase, once the appropriate cell density was reached (above 7×10⁶ cells/ml), cells were inoculated with Influenza virus (Multiplicity of Infection of 1×10⁻⁶) in perfusion mode and the temperature was switched to 33° C. The virus was harvested by collecting the cell culture medium by perfusion at days 3 and 4 post-inoculation (JP115) or at days 3, 4 and 5 post-inoculation (NCP117). The perfusion harvests were pooled and stored at a temperature ranging from 2 to 8° C. until further processing.

### 2. Virus Isolation

a) The viral harvest was clarified on a filtration train composed of three different depth filters with nominal porosities of 5 μm-0.5 μm-0.2 μm. The clarified harvest was stored at a temperature ranging from 2 to 8° C. overnight.

b) The clarified harvest was then concentrated ten fold by ultrafiltration with a 750 kD hollow fiber membrane, diafiltrated against 5 volumes of PBS containing 125 mM citrate and 0.001% Triton X-100 pH 7.4 and against 4 volumes of 10 mM Tris, 2 mM MgCl₂, 100 μM CaCl₂, 0.001% Triton X-100 pH 8.

c) The retentate was removed from the ultrafiltration system and warmed up to 37° C. in a water bath. DNA degradation was performed by adding BENZONASE™ (Merck) to the retentate at a final concentration of 270 Units/ml (JP115) or 135 Units/ml (NCP117) and the mixture is incubated 1 hour at 37° C.

d) Next, the ultrafiltration retentate was subjected to a sucrose gradient (0-55%) ultracentrifugation, using a 400 ml rotor, wherein virus and contaminants migrate into the gradient until reaching their respective density. Once all the retentate was loaded onto the gradient, a banding time of 60 minutes allows most of the virus to reach its density within the gradient. The viral particles were concentrated within a few fractions. The product fractions were in PBS pH 7.4 containing 125 mM citrate and sucrose. The purified whole virion was pooled from the percentage of sucrose ranging from approximately 28 to 50%. This range has been determined on the basis of profiles from SDS-PAGE and from Western Blot analysis using anti-HA and anti-MDCK antibodies. The whole virion pooled fractions were stored at a temperature ranging from 2 to 8° C., then diluted 9-10 fold in PBS pH 7.4 (NCP117) or PO₄ 66 mM pH 7.4 (JP115).

e) A second sucrose gradient ultracentrifugation was performed in order to further purify the virus, while simultaneously splitting it. A combination of 1% Triton X-100, 1% Deoxycholate and 0.5 mM alpha-tocopheryl hydrogen succinate (JP115) or of 1% Triton X-100 and 0.5% Sodium lauryl sulfate (NCP117) was added to the sucrose layers to achieve a detergent micelles barrier. The whole virus entering this detergent barrier was split. Virus fragments containing the viral membrane proteins hemagglutinin (HA) and neuraminidase (NA) migrated to the micelles density. The remaining virions, some of the host cell protein contaminants and DNA migrated to higher sucrose concentration fractions which are not pooled with the viral proteins. The viral proteins present in the fractions ranging from approximately 15 to 40% sucrose were pooled. This range has been determined on the basis of profiles from SDS-PAGE and from Western blot analysis using anti-HA and anti-MDCK antibodies. The fractions pool containing the viral proteins were in PBS pH 7.4. This pool was then assayed for the total protein content and diluted to 250 μg protein/ml with PBS containing 0.01% TWEEN™ 80 and alpha-tocopheryl hydrogen succinate 0.1 mM pH 7.4 (NCP117) or with PO₄ 66 mM pH 7.4 (JP115).

f) Formaldehyde was added to detergent-inactivated pool of viruses to further inactivate the virus. Formaldehyde is added at a ratio of 50 μg for 250 μg total proteins. A 0.2 μm sterilizing grade filtration was performed immediately after formaldehyde addition. Incubation lasts 72 hours at room temperature in sterile conditions.

g) The product from the formaldehyde inactivation was supplemented with 200 mM NaCl (JP115) or 250 mM (NCP117), filtered through an anion exchange membrane in order to eliminate residual DNA, which binds to the membrane. The addition of NaCl prevented the product from binding to the membrane, said product being thus collected in the membrane flow-through.

h) Biobeads-SM2 microporous divinyl-benzene cross-linked polystyrene with a high specific area for detergent adsorption were added in batch to the anion exchange membrane flow-through, which was incubated for 1 hour at room temperature under stirring. At the end of this step, the Biobeads-SM2 were removed.

i) The viral proteins were then concentrated and diafiltered against PBS pH 7.4 containing 0.01% TWEEN™ 80 and 0.1 mM α-tocopheryl hydrogen succinate using a 30 kD cellulose regenerated flat sheet Hydrosart membrane (Sartorius) to reduce the residual sucrose and formaldehyde amounts.

j) The resulting bulk was filtered onto a 0.2 μm membrane in a class 100 aseptic area. If needed, the bulk was diluted during filtration on PBS pH 7.4 containing 0.01% TWEEN™ 80 and 0.01% Triton X-100 to adjust total protein concentration to approximately 450 μg/ml. The resulting sterile product was referred to as the monovalent purified bulk.

### Example 2

### Production of Influenza Virus in MDCK Cells in the Presence of Multiple Steps of DNA Degradation Performed with an Endonuclease and a DNA Alkylating Agent

### 1. Virus Multiplication

The MDCK adherent cells were grown on microcarriers in a perfusion culture mode in a 20 liter stirred bioreactor scale at 36.5° C. After the growth phase, once the appropriate cell density was reached (above 7×10⁶ cells/ml), cells were infected with Influenza virus (Multiplicity of Infection of 1×10⁻⁵) in a perfusion mode and temperature was switched to 33° C. BENZONASE™ (Merck) was added according to one of the following conditions:

(i) at a final concentration of 1.5 Units/ml in the perfusion at days 3 and day 4 post-inoculation (JP125—Jiangsu B strain, NCP127—New Caledonia A strain, DFC1AFA002—New Caledonia A strain, DFC2AFA001—New York A strain and DFC3APA002—Jiangsu B strain),
(ii) at a final concentration of 1 Unit/ml in the bioreactor at day 1, 2, 3 and 4 post-inoculation (B005—New York A strain),
(iii) during the day 4 of perfusion in order to obtain a final concentration of 3 Units/ml in the harvest pool (JP128—Jiangsu B strain).

The virus was harvested by collecting the cell culture medium by perfusion at days 3, 4 and 5 post-inoculation for conditions (i) and (ii) and at day 2, 3, 4 and 5 for condition (iii). The perfusion harvests were pooled and stored at 4° C. until further processing.

In condition (iv), BENZONASE™ (Merck) was added to the viral harvest at a final concentration of 10 Units/ml (NCP124-New Caledonia A strain), after pooling the perfusion harvests of day 3, 4 and 5 post-inoculation. The resulting mixture was incubated at room temperature during 1 hour.

### 2. Virus Isolation

The different viral harvests were clarified and subjected to ultrafiltration as in Example 1 according to the steps a) and b), except that the NCP127 viral harvest was added with TWEEN™ 80 at a final concentration of 0.02% before clarification.

c) All retentates were removed from the ultrafiltration system and warmed up to 37° C. in a water bath. DNA degradation was performed by adding BENZONASE™ (Merck) to the retentate at a final concentration of 100 Units/ml (NCP124), 200 Units/ml (JP125 and DFC3APA002) or 135 Units/ml (NCP127, JP128, DFC1AFA002 and DFC2AFA001) and the mixtures were incubated for 1 hour at 37° C.

The rest of the isolation was performed as described in Example 1, in accordance with steps d) to j), with the following modifications:
A step of beta-propiolactone (BPL) treatment was implemented, either before (NCP124, NCP127, DFC1AFA002, DFC2AFA001 and DFC3APA002) or immediately after step d) (JP125 and JP128) during which BPL was added at a final concentration of 0.05% (NCP124, DFC1AFA002, DFC2AFA001 and DFC3APA002) or 0.1% (JP125) and incubated overnight at a temperature ranging from 2 to 8° C. (JP125 and NCP124) or at room temperature (DFC1AFA002, DFC2AFA001 and DFC3APA002).
The NCP124 sample also had a first step of BPL treatment before the second BENZONASE™ degradation, immediately after clarification step a) and before the ultrafiltration step b), wherein BPL was added at a final concentration of 0.05% and incubated overnight at a temperature ranging from 2 to 8° C.
The whole virus from NCP124 sample, at the step e), was split in the presence of a mixture of Triton X-100 1.5%+Sodium Lauryl Sulfate 1%.
After the splitting ultracentrifugation of step e), the samples DFC1AFA002, DFC2AFA001 and DFC3APA002 were filtrated, supplemented with 0.3% of Triton X-100 and left stored at room temperature for 72 hours before further processing, with step f).

### Example 3

### Methods for Measuring the Size and the Amount of Residual DNA

1. DNA Sample Treatment for DNA from MDCK Cells

Viral harvests or purified bulks (30 ml), as indicated, were first treated with 100 μg/ml proteinase K in the presence of 0.1% SDS, at 55° C. overnight, followed by a standard phenol/chloroform extraction. The resulting solution was then concentrated using a Centriplus centrifugal filter device (Millipore, YM-30 4422). Next, the sample was treated with 5% RNase (Roche) for 90 minutes at 37° C., followed by a second concentration step using a Microcon centrifugal filter device (Millipore, YM-50 42416). The final volume was split into two fractions, the first one aimed at DNA visualization by agarose gel and Southern-blot analysis and the second one intended for a Q-PCR quantification.

2. DNA Sample Treatment for DNA from EB66™ Cells

Purified bulks (4 ml) were treated with 200 μg/ml proteinase K in the presence of 0.1% SDS, at 55° C. for 2 hours. The DNA is then extracted with Extraction Nuclisens Magnetic Kit (BioMérieux) according to the manufacturer's instructions.

3. Agarose Gel Applicable to DNA from MDCK Cells and from EB66™ Cells

DNA samples were loaded on a 1.25% agarose gel containing Ethidium Bromide and DNA was visualized on UV light. Pictures are taken.

4. Q-PCR Specifically Designed for Amplifying DNA from MDCK Cells

### a) Design:

A quantitative PCR (Q-PCR)-based approach designed to specifically amplify and quantify two highly repetitive canine sequences of 63 base pairs (bp) and 314 bp, respectively, has been developed. These two sequences are the Short Interspersed Repetitive DNA elements (SINE) and the Long Interspersed Repetitive DNA elements (LINE), respectively. The primers and the probe used to amplify LINE and SINE sequences are listed in Table 1.

TABLE 1
Primers used for amplifying LINE and SINE
sequences from MDCK cells

| Primers | Sequence |
|---|---|
| Canis-Sine-63TMF | 5′-ATCCTGGAGTCCTGGGATCGA-3′ |
| Canis-Sine-63TMR | 5′-AGAGGGAGAAGCAGGCTCCAT-3′ |
| Canis-Sine-MGBprobe | 5′-FAM-CCCACATCGGGCTC-DQ-MGB-3′ |
| Canis-Line-314TMF | 5′-GGAAGGAGCCTCGGTGTCCAWC-3′ |
| Canis-Line-314TMR | 5′-CCACCCCTAGTTCGTTTCCCAGAG-3′ |

The SINE Q-PCR is a TaqMan based PCR using a fluorescent minor groove binding (MGB) probe while the LINE Q-PCR is a Sybergreen based PCR. These two PCR are expected to generate amplicons of 63 and 314 bp, respectively.

To develop appropriate positive controls for establishment of quantification curves by Q-PCR, the genomic DNA of MDCK cells was extracted and purified by a Qiagen genomic extraction kit (ref 13362) according to the manufacturer's recommendations.

The extracted DNA was then either double digested with the restriction enzymes EcoRI/XhoI, or digested with Pst1. The resulting DNA fragments were then quantified by the THRESHOLD™ assay (Molecular Devices Corporation) (see section 4) according to the manufacturer's recommendations.

These positive controls were used as described below to establish standard curves to quantify residual MDCK DNA but also in validation experiments and experiments designed to detect PCR inhibitions.

Samples were then added to the TaqGold DNA polymerase kit mixture (Applied biosystems) to reach a final volume of 50 μl. PCR reactions were performed on an ABI 7900 thermocycler and started with a first step of 2 min at 50° C. followed by a denaturation step at 95° C. for 10 min and then by 40 cycles consisting of a first step of 15 sec at 94° C. followed by a second step at 60° C. for 1 min.

### b) Detection of Q-PCR Inhibitions:

To assess that no PCR inhibition takes place during the Q-PCR experiments, each Q-PCR was performed in duplicate. In one of these two reactions, a known amount of digested MDCK DNA was spiked and used to estimate Q-PCR inhibitions. Inhibition was calculated and used as a value to correct the SINE and LINE Q-PCR quantitative data.

### c) Determination of the DNA Recovery:

The impact of the extraction/concentration procedure (see section 1. DNA sample treatment) on the efficiency of DNA fragments recovery was assessed by spiking samples with four external DNA amplicons of various lengths: (i) a 659 bp amplicon from rhinovirus 5′NC sequence, (ii) a human 305 bp amplicon from the M gene of the parainfluenza 1 virus, (iii) a 138 bp amplicon from the hexon gene of adenovirus BC, and (iv) a 87 bp amplicon from the gp2 gene of the human polyoma virus genome. These external DNA amplicons (100 ng each) were added to 30 ml of purified bulks. In parallel to the Q-PCR amplifying specific canine LINE and SINE sequences, Q-PCRs targeting the 4 different external DNA were performed to determine the DNA recovery of the 4 fragments spiked. Recovery was calculated and used as a value to correct the SINE and LINE Q-PCR quantitative data.

### d) Q-PCR Validation:

The efficiency, repeatability, sensitivity and specifity of both Q-PCR were determined. Serial 10-fold dilutions (ranging from 0.01 pg to 10³ pg) of MDCK DNA double digested with EcoRI/XhoI or digested with Pst1 were used in these experiments. Dilutions were prepared by two technicians and each dilution was tested in duplicate allowing to determine the efficiency, repeatability and sensitivity of both Q-PCR. The specificity was determined by loading amplicons resulting from both Q-PCR on agarose gel to ensure that a unique band of the expected size can be observed.

Results—Conclusions

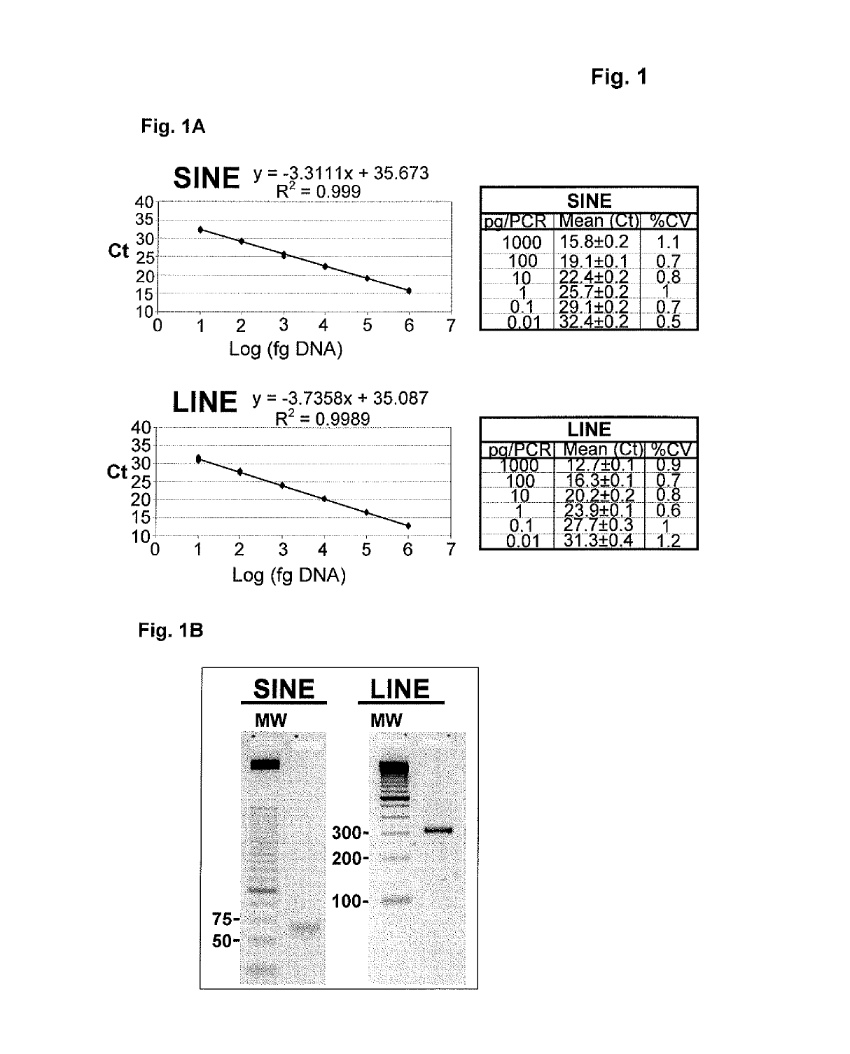

The results, as presented in FIG. 1A, demonstrated excellent linearity from which efficiencies of 100% and 85% were calculated for SINE and LINE Q-PCR, respectively. R² values of 0.999 and 0.9989, reflecting repeatability, were calculated for SINE and LINE Q-PCR, respectively, and it was determined that the sensitivity of both tests is 10 fg of DNA, i.e. an amount of DNA of less than 10 fg within the sample to be tested will not be detected.

To demonstrate that the fluorescent signals generated during the Q-PCR were specific for amplification of the targeted SINE and LINE sequences, Q-PCR products were loaded on agarose gel to demonstrate the presence of the expected 63 bp (SINE Q-PCR) and 314 bp (LINE Q-PCR) fragments. Results are presented in FIG. 1B and allowed to clearly visualize a unique band of the expected molecular weight for both Q-PCR.

Since the Q-PCR developed here are designed to detect very low amounts of residual DNA within samples resulting from the extraction/concentration procedure, it is of crucial importance to investigate if this procedure could impact the DNA recovery. From all the spiking experiments, as described above, that were performed, it was calculated that fragments of 138 bp, 305 bp and 659 bp were detected at a recovery rate of approximately 50% (mean calculated on the recovery rate obtained from the different experiments), while the fragment of 87 bp was detected at a recovery rate less than 25%.

5. Q-PCR Specifically Designed for Amplifying DNA from EB66™ Cells

A quantitative PCR (Q-PCR)-based approach designed to specifically amplify and quantify duck sequences of 99 base pairs (bp), 185 base pairs (bp) and 280 base pairs (bp) by using a common probe (P123), a common forward primer (TMF123), and three different reverse primers (TMR100, TMR200 and TMR300) (see FIG. 10) has been developed. These three sequences are located within a Long Interspersed Repetitive DNA element (LINE) of the duck genome. The primers and the probe used to amplify the three sequences are listed in Table 2.

TABLE 2
Primers used for amplifying the sequences of 99,
185 and 280 bP from EB66™ cells

| Primers | Sequences |
|---|---|
| TMF123 | 5′-GGGCGACCTTATCGCTCTCT-3′ |
| TMR100 | 5′-TTCCCCCTCGTCCTGTCA-3′ |
| TMR200 | 5′-GCCTGTTCCAGTGYCTAACAA-3′ |
| TMR300 | 5′-CACTAACAGTCCCCACTAAACCATATC-3′ |
| P123 | 5′-FAM-TTGGCCTGTTCTCCCACGTGCC-EDQ-3′ |

Q-PCR uses a fluorogenic probe to enable the detection of a specific PCR product as it accumulates during PCR cycles. The fluorogenic probe consists of an oligonucleotide with a covalently linked 5′-reporter dye and a 3′-quencher dye. During the PCR reaction, the 5′ exonuclease activity of Taq DNA polymerase cleaves the fluorogenic probe. The 5′ fluorescent reporter dye is released and the accumulation of PCR product can be detected by measurement of the Fluorescence. The rise of sequence generation is monitored in real-time and data are collected throughout the PCR by the software of an Applied Biosystems (ABI) 7900HT thermocycler. Absolute quantification of the residual host cell DNA can be calculated using a standard curve generated from known quantities of genomic EB66™ DNA. For that purpose, genomic DNA from 10⁸ EB66™ cells was extracted by Blood and cells culture DNA maxi kit (Qiagen), with genomic-tip G500 (Qiagen) following the manufacturer's instructions and DNA was eluted in sterile water. DNA was then double-digested with the restriction enzymes Pst1/Ssp1. After digestion, DNA was purified by a classical phenol/chloroform treatment, followed by an ethanol precipitation step. DNA was then eluted in water, and quantified by the THRESHOLD™ assay (see section 5). The standard curve was established by performing Q-PCR reactions on serial 10-fold dilutions starting from 1 ng to 10 fg of digested genomic EB66™ DNA.

In order to establish the repeatability and sensitivity of the three Q-PCR reactions, dilutions of double digested and quantified EB66™ genomic DNA were prepared as described above. Q-PCR for amplifying the three sequences of 99 bp, 185 bp and 280 bp were performed independently and in duplicate on each dilution. PCR reactions were performed on an ABI 7900 thermocycler and started with a first step of 2 min at 50° C. followed by a denaturation step at 95° C. for 10 min and then by 40 cycles consisting of a first step of 15 sec at 94° C. followed by a second step at 60° C. for 1 min. The results obtained for each Q-PCR reaction are presented in Table 3.

TABLE 3
Q-PCR performed on EB66 ™ standard DNA

| Digested | | | | | | |
|---|---|---|---|---|---|---|
| EB66 ™ | | | | | | |
| DNA | Q-PCR 99 bp | | Q-PCR 185 bp | | Q-PCR 280 bp | |
| (fg/PCR) | Ct1** | Ct2 | Ct1 | Ct2 | Ct1 | Ct2 |
| 100000 | 19 | 19 | 20 | 19 | 19 | 20 |
| 100000 | 22 | 23 | 23 | 23 | 23 | 23 |
| 10000 | 26 | 26 | 26 | 26 | 27 | 27 |
| 1000 | 29 | 29 | 30 | 30 | 30 | 30 |
| 100 | 33 | 33 | 34 | 34 | 34 | 34 |
| 10 | 35 | 37 | 37 | 37 | 37 | 38 |
| Blank PCR* | 39 | 40 | 40 | 40 | 40 | 40 |

| | | |
|---|---|---|
| Y = −3.4997x + | Y = −3.462x + | Y = −3.6185x + |
| 40.001 | 40.255 | 41.181 |
| R² = 0.9994 | R² = 0.9993 | R² = 0.9999 |

*means that no DNA was present in the PCR tube and serves as negative control
**Ct is for THRESHOLD ™ Cycle

Results—Conclusions

The three Q-PCR exhibit the same linearity and the same sensitivity. R² values of 0.9994, 0.9993 and 0.9989, reflecting repeatability, were calculated for 99 bp, 185 bp and 280 bp sequences, respectively, and it was determined that the sensitivity of the three tests is 10 fg of DNA, i.e. an amount of DNA of less than 10 fg within the sample to be tested will not be detected.

To assess that no PCR inhibition takes place during the Q-PCR experiments, each Q-PCR was performed in duplicate. In one of these two reactions, a known amount of digested EB66™ DNA was spiked and used to estimate Q-PCR inhibitions. Inhibition, if any, was calculated and used as a value to correct the Q-PCR quantitative data. The impact of the extraction/concentration procedure (see section 2. DNA sample treatment) on the efficiency of DNA fragments recovery was assessed by spiking the purified bulks to be tested before extraction/concentration with known amounts of external DNA amplicons of various lengths. DNA was then extracted from the spiked bulks and concentrated. In parallel to the Q-PCR amplifying specific sequences of 99 bp, 185 bp and 280 bp, Q-PCRs targeting the different external DNA amplicons were performed to determine the DNA recovery of the fragments which were spiked. Recovery was calculated and used as a value to correct the Q-PCR quantitative data obtained for the 99 bp, 185 bp and 280 bp sequences.

6. Southern-Blot/Phospholmager for Analysing DNA from MDCK Cells

Concentrated DNA was loaded on agarose gel and fragments were transferred to a nylon membrane by capillary transfer. In order to prepare the probes for detecting transferred residual DNA fragments, extracted MDCK DNA was digested with the restriction enzyme Sau3A following the manufacturer's recommendations. The resulting fragments were labeled with α-[³²P] dCTP using a random-primed labeling kit (Roche). Membranes were hybridized at 50° C. overnight and then washed, dried and used to expose an X-ray film. For Phospholmager semi-quantification, serial dilutions of known amounts of Sau3 digested MDCK DNA, as measured by THRESHOLD™ assay, were also subjected to Southern-blot analysis. A radiosensitive screen exposed with the hybridized membranes was analyzed using a Phospholmager (Storm 860, Amersham Biosciences) with the ImageQuant TL analysis software (Amersham).

7. THRESHOLD™ Assay Applicable for DNA Analysis from MDCK Cells and from EB66™ Cells

The THRESHOLD™ system from Molecular Devices is a quantitative assay for picogram levels of total DNA, and has been used for monitoring levels of contaminating DNA in biopharmaceuticals products (Briggs, J. and Panfili, P. R., 1991, Anal. Chem. 63(9): 850-859). A typical assay involves non-sequence-specific formation of a reaction complex between a biotinylated single stranded DNA binding protein, a urease-conjugated anti-single stranded DNA antibody, and DNA. All assay components are included in the complete Total DNA Assay kit available from the manufacturer and the reaction is performed according to the manufacturer's recommendations. Briefly, after DNA denaturation by boiling, the samples to be tested are combined, in liquid phase, with a single reagent containing conjugates of two DNA binding proteins plus streptavidin. A monoclonal anti-DNA antibody is conjugated directly to urease; E. Coli single-stranded binding protein is conjugated to biotin. Both binding proteins have high affinity for single-stranded DNA with weak sequence specificity. Streptavidine is used for the specific capture of the complexes formed onto a biotinylated membrane and urease is used for enzymatic signal generation. During the liquid-phase incubation, complexes containing DNA, streptavidin and urease are formed. These complexes are then captured on a biotinilylated membrane by filtration. After washing the membrane, the amount of urease retained on the membrane is quantitatively related to the amount of DNA in the sample. The signal detection occurs by contacting the captured complexes-containing membranes with a solution of urea in the THRESHOLD™ reader.

### Example 4

### Comparison of Residual MDCK Cells DNA Profile Present in Influenza Virus Harvest Obtained by Collecting the Cell Culture Supernatant of Infected Cells—One DNA Degradation Step Performed with an Endonuclease During the Virus Multiplication Phase Vs No DNA Degradation Step

In order to evaluate the effect of implementing one step of DNA degradation during the virus multiplication phase, harvests obtained according to Example 1 (no DNA degradation step; JP115 and NCP117) and Example 2 (one DNA degradation step; JP125) were either directly loaded on an agarose gel or DNA samples were prepared, as specified in Example 3, for a Q-PCR analysis.

DNA profile was analyzed by (i) agarose gel and (ii) by Q-PCR, as specified in section 3 and 4 of Example 3.

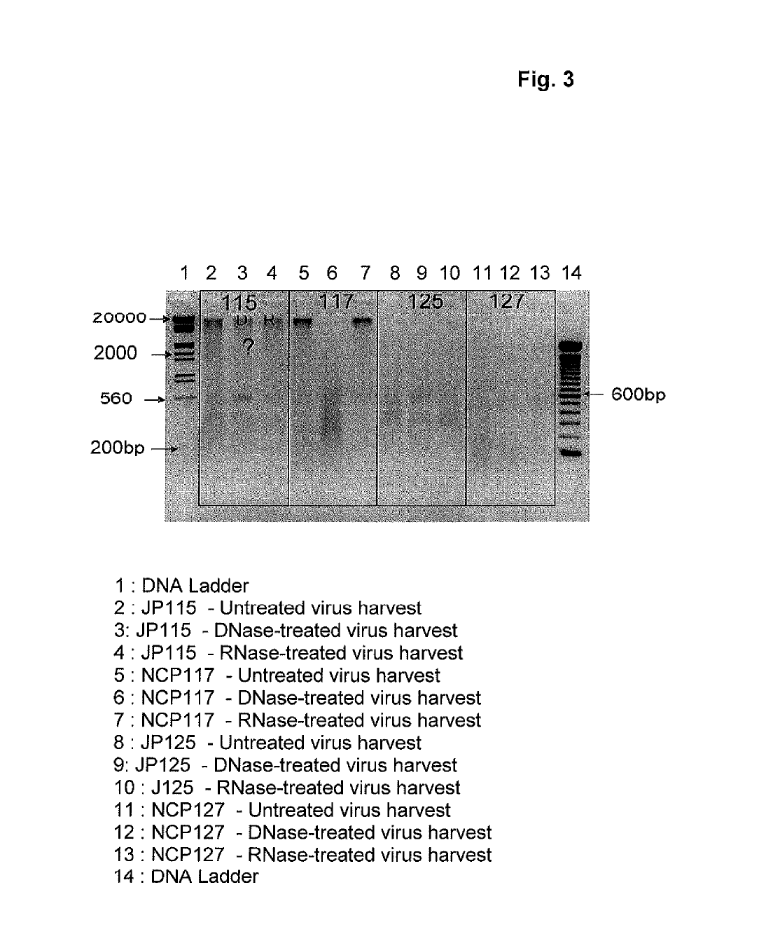

Pictures of agarose gel are shown in FIG. 3.

Data obtained by Q-PCR are listed in Table 4.

TABLE 4
DNA concentration in viral harvests measured by Q-PCR

| | Q-PCR results | | | |
|---|---|---|---|---|
| | Viral Harvest | | | |
| | JP115 | | JP125 | |
| | SINE | LINE | SINE | LINE |
| Probe | 63 bp | 314 bp | 63 bp | 314 bp |
| DNA concentration (μg/ml) | 5 | 3 | 2 | 0.5 |
| DNA fold decrease | — | — | 2.5 | 6 |
| DNA content (μg/45 μg HA) | — | — | 0.36 | 0.09 |

Results—Conclusions

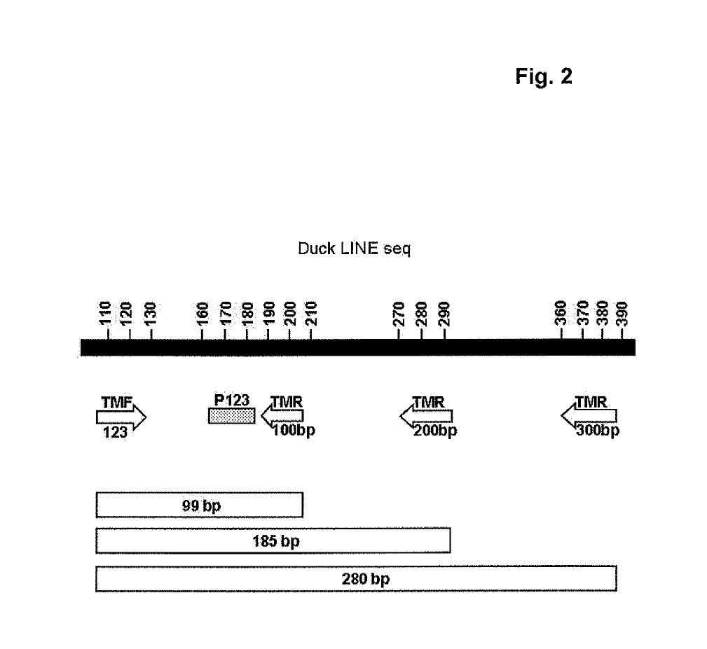

The agarose gel presented in the FIG. 3 shows a high DNA content with mainly an intense DNA band at a size of approximately 20 000 bp in lanes where no BENZONASE™ step has been carried out, i.e. in JP115 sample (see lane 2 of the gel presented in FIG. 3) and NCP117 sample (see lane 5 of the gel presented in FIG. 2).

In case of examples where one step of BENZONASE™ treatment has been performed during the virus multiplication phase, the gels show a much lower DNA content and size with a smear or faint bands between 200 and 560 bp, i.e. in JP125 sample (lane 8) and NCP127 sample (lane 11).

The results obtained by Q-PCR show a significant DNA content reduction in the viral harvest resulting from a cell culture treated with BENZONASE™ (JP125), as opposed to the viral harvest resulting from a cell culture not treated with BENZONASE™ (JP115): the addition of BENZONASE™ results in a 2.5-fold (JP125) decrease for the 63 bp long DNA and a 6-fold (JP125) decrease for the 314 bp long DNA. With such DNA fold decrease values, the method according to the invention allows to reduce 63 bp long DNA and 314 bp long DNA by 60% and more than 80%, respectively, before proceeding to the virus isolation phase. If measuring in parallel the HA amount present within the same viral harvests by SRD assay (see Example 9), then it is possible to normalize the DNA amount against 45 μg HA, which is the expected regular HA amount for a trivalent vaccine containing 15 μg HA of each strain (last row of Table 4).

Therefore, the BENZONASE™ step during the virus multiplication phase (i) has a positive impact on the DNA size with the observation of a strong reduction of the intensity of the DNA bands higher than 20 000 bp; (ii) reduces significantly the DNA content with a reduction ranging from 2.5 fold to 6 fold depending on the probe used; (iii) increases the virus purity regarding the contaminating DNA; and (iv) increases the safety of the vaccine due to the reduction of the contaminating level of and the size of DNA.

### Example 5

### Comparison of the Profile of Residual MDCK Cells DNA after One DNA Degradation Step Vs Two DNA Degradation Steps Performed with an Endonuclease

In order to evaluate the effect of implementing two steps of DNA degradation, one during the virus multiplication phase and one during the virus isolation phase, DNA samples were prepared, as specified in Example 3, before and after the DNA degradation occurring at the step c) of the virus isolation process described in Example 1 and 2, so that DNA concentration could be determined through Q-PCR analysis, allowing thus calculating a DNA log reduction.

DNA profile was analyzed by Q-PCR, as detailed in section 4 of Example 3. Results are presented in Table 5.

TABLE 5
DNA log reduction

| | Q-PCR (DNA log reduction) | | | |
|---|---|---|---|---|
| Steps/Samples | JP115 | | JP125 | |
| BENZONASE ™ during the | − | | + | |
| virus multiplication phase | | | | |
| Clarification | + | | + | |
| 750 kD Ultrafiltration | + | | + | |
| BENZONASE ™ in the | + | | + | |
| ultrafiltration retentate | | | | |
| | SINE | LINE | SINE | LINE |
| Probe | 63 bp | 314 bp | 63 bp | 314 bp |
| DNA log reduction | 2 log | 1 log | 2-3 log | 2-3 log |

Results—Conclusions

For the JP115 experiment, the BENZONASE™ treatment of the ultrafiltration retentate induced a DNA log reduction of 2 log with respect to 63 bp long fragments and 1 log with respect to 314 bp long fragments.

For the JP125 experiment, in which BENZONASE™ was additionally added during the viral multiplication phase, the DNA log reduction ranges from 2 to 3 logs for both probes, 63 bp and 314 bp. Accordingly, the BENZONASE™ treatment of the ultrafiltration retentate induced a higher DNA log reduction when a first DNA degradation has been previously performed during the virus multiplication phase. It is to be noted, also, that the log reduction is more important regarding the 314 bp probe.

Therefore, a DNA degradation step during the virus multiplication phase allows for a far more effective DNA degradation during the DNA degradation step after the ultrafiltration.

### Example 6

### Comparison of the Amount of Residual MDCK Cells DNA in the Purified Bulk of Cell Culture-Produced Influenza Viruses—Multiple DNA Degradation Steps Vs One DNA Degradation Step

In order to evaluate the impact of implementing multiple DNA degradation steps on host cell DNA elimination, DNA content was measured at the end of the virus isolation process of samples JP115 and JP125, i.e. in the corresponding purified bulks. The DNA content was analysed by the classical THRESHOLD™ assay, as specified in section 7 of Example 3, as presented in Table 6.

TABLE 6
DNA amount present in purified bulks

| | JP115 | JP125 | DFC1AFA002 | DFC2AFA001 | DFC3APA002 |
|---|---|---|---|---|---|
| DNA (ng/45 μg HA) | 0.7 | <0.21 | <0.2 | 0.42 | 0.40 |

Results—Conclusion

In order to obtain DNA amounts expressed in ng/45 μg HA, the HA content in the purified bulks was determined in parallel, via SRD assay (see Example 9), and the DNA values obtained were normalized against 45 μg HA, the expected regular dose of a trivalent vaccine containing 15 μg HA of each strain.

Therefore, the final DNA amount measured in the purified bulk of JP115 was 0.7 ng, while the purified bulk of JP125 presented less than 0.21 ng of total DNA. These values are significantly lower than the limit of 10 ng/dose specified by the authorities. Moreover, they indicate that the implementation of multiple DNA degradation steps, i.e. two steps with an endonuclease and one step with a DNA alkylating agent (JP125) allows to get a 3.3-fold reduction of total DNA in the purified bulk, as compared to the implementation of only one degradation step performed with an endonuclease (JP115).

The experiments DFC1AFA002, DFC2AFA001 and DFC3APA002 represent additional experiments of multiple DNA degradation steps based on the combination of two endonuclease steps and one DNA alkylating agent step, similarly to JP125. Measurement of the final DNA amount in the purified bulks gave similar low amounts ranging from 0.2 to 0.40 ng/45 μg HA, as compared to JP115.

### Example 7

### Comparison of the Size Distribution of Residual MDCK Cells DNA in the Purified Bulk of Cell Culture-Produced Influenza Viruses—Multiple DNA Degradation Steps Vs One DNA Degradation Step

In order to get more information about the size of the residual host cell DNA still present in the purified bulk of cell culture-produced Influenza viruses, DNA samples were prepared from 30 ml of purified bulks originating from samples JP115, JP125, NCP124, DFC1AFA002, DFC2AFA001 and DFC3APA002, as described in section 1 of Example 3. Their residual host cell DNA profile was analyzed according to the following methods, as indicated.

DNA size was analyzed by Southern-blot analysis and Phospholmager semi-quantification, as described in section 6 of Example 3.

As an alternative and independent assay, a Q-PCR was also made on the same DNA sample with the primers SINE and LINE, as specified in section 4 of Example 3, in order to quantify, respectively, residual fragments of 63 bp and of 314 bp length. As indicated on section 4b) and 4c) of the Example 3, the obtained values were corrected in light of the data of the recovery and inhibition experiments.

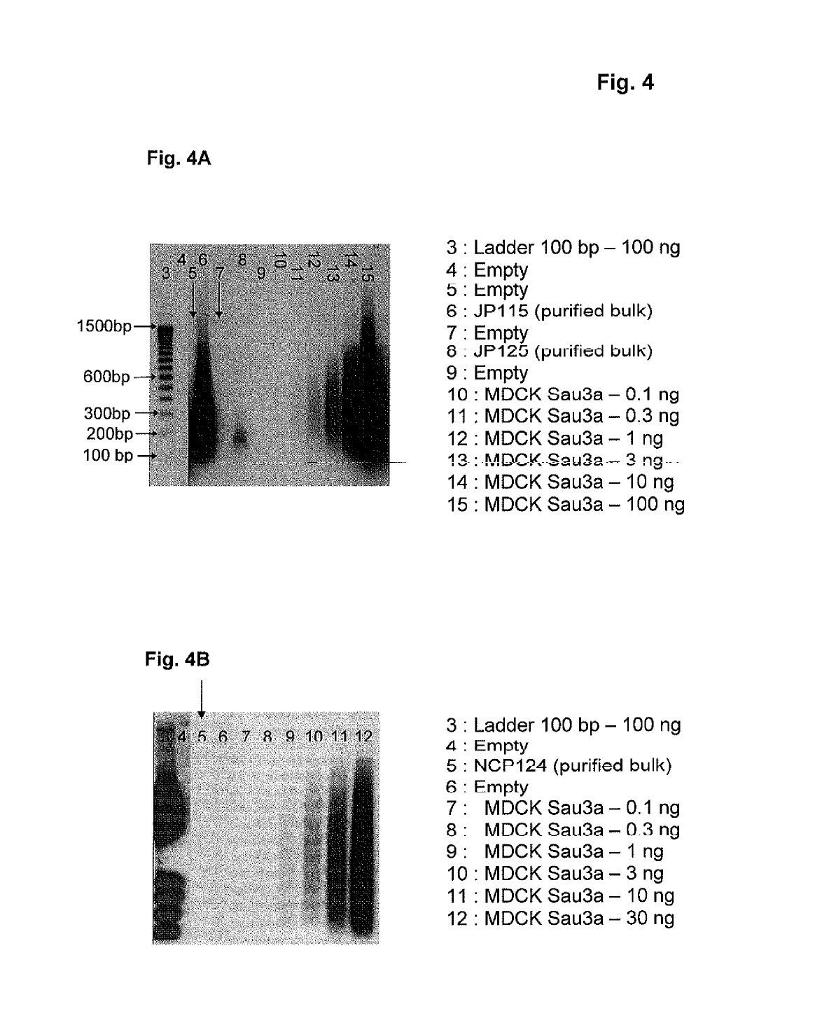

Southern-blot pictures are presented in FIG. 4. Results of the Southern-blot/Phospholmager semi-quantification and the Q-PCR analysis are presented in Table 7.

TABLE 7
DNA amount and size in purified bulks

| | Southern- | | | |
|---|---|---|---|---|
| | Blot/ | | | |
| | Phosphoimager | | Q-PCR | |
| | DNA size | | | |
| | | | SINE | LINE |
| | 100-300 bp | >300 bp | 63 bp | 314 bp |
| JP125 (ng/45 μg HA) | 0.011 | <0.001 | 0.007 | <0.001 |
| JP115 (ng/45 μg HA) | 0.33 | 0.18 | 0.56 | 0.34 |
| DFC1AFA002 | 0.003 | NQ* | 0.003-0.008 | <0.001 |
| (ng/45 μg HA) | | | | |
| DFC2AFA001 | 0.005 | 0.001 | 0.015-0.05 | <0.001 |
| (ng/45 μg HA) | | | | |
| DFC3APA002 | 0.027 | 0.006 | 0.0027-0.065 | <0.001 |
| (ng/45 μg HA) | | | | |

*NQ = non quantitable

Results—Conclusion

From the Southern-blot pictures, in FIG. 4, it can be deduced visually that, when only one BENZONASE™ step is performed in the process, during the virus isolation phase, as for the JP115 sample, a high amount of residual DNA (see lane 6 of the scan presented on FIG. 4A) is detected, with lots of fragments above 300 bp.

However, when two BENZONASE™ steps and one BPL step were combined, the level of residual host cell DNA and the size of the fragments were much lower as can be seen for the JP125 sample (see lane 8 of the scan presented in FIG. 4A). Only traces of fragments ranging from 100-200 bp long were detectable.

This low DNA content has also been observed in other samples including two BENZONASE™ steps and one BPL step, such as in sample NCP124 (see lane 5 of the scan presented on FIG. 4B).

The semi-quantitation by Phospholmager indicated that the amount of residual DNA fragments ranging from 100 to 300 base pairs ranged from 0.003 to 0.027 ng/dose (Table 7, first column). The DNA dose amount was obtained by determining in parallel the HA content via SRD assay (see Example 9) in the purified bulks. Then, the DNA values per dose were normalized against 45 μg HA, the expected regular dose of a trivalent vaccine containing 15 μg HA of each strain. The values obtained were much lower than the limit specified by the authorities (10 ng/dose), as already measured by THRESHOLD™ assay. The quantitation of residual host cell DNA fragments upper than 300 base pairs indicated that this population of DNA fragments sizes were rare since they were either not detectable (DFC1AFA002) or ranging from 0.001 to 0.006 ng/dose (Table 7, second column). These results demonstrate the efficiency of the process to reduce residual host cell DNA size to fragments sizes mainly lower 300 to 200 base pairs. If comparing the sample JP115, where only one step of DNA degradation was implemented, with the sample JP125, where two steps of DNA degradation were implemented, it is observed a much lower DNA amount when two steps are performed, whether considering fragments bigger than 300 bp or fragments ranging from 100 to 300 bp. Indeed, a 180-fold reduction was observed for DNA fragments bigger than 300 bp, 0.18 ng (JP115) versus <0.001 ng (JP125), and a 30-fold reduction was observed for DNA fragments ranging from 100 to 300 bp, 0.33 (JP115) ng versus 0.011 ng (JP125).

The Q-PCR analysis targeting residual DNA fragments of 63 base pairs and 314 base pairs, SINE and LINE sequences, respectively, showed that the 63 base pairs fragments were detected at levels ranging from 0.003 to 0.065 ng/dose (Table 7, third column). The 314 base pairs fragments were in most cases below the detection limit of the Q-PCR test. The detection limit was 10 fg which need to be initially present in the PCR tube (Table 7, last fourth column). In the context of the present experiment, such a minimal value corresponds to a minimal value of 0.001 ng/45 μg DNA. As observed with the Southern-blot experiment, if comparing JP115 (one DNA degradation step) with JP125 (two DNA degradation steps), a strong reduction of the DNA amount was observed when two steps were implemented, whether considering the 60 bp long fragments or the 300 bp long fragments. Indeed, a 340-fold reduction (0.34 versus 0.001) was observed for 300 bp long DNA fragments and a 80-fold reduction (0.56 versus 0.007) was observed for 60 bp long DNA fragments. These results also indicate, as observed in Southern-blot experiments, a strong reduction in DNA size. Indeed, these low values are corroborated by the values obtained through Phospholmager semi-quantitation.

Therefore, the characterization of the residual DNA in purified bulk demonstrated that the production process is able not only to readily reduce total DNA amount from the process to very low levels (the amount of residual DNA is in the range of pg/dose), but also to degrade this DNA to low size fragments (below 300 bp), which further ensures the safety of the vaccine.

### Example 8

### DNA Clearance after Multiple DNA Degradation Steps Performed with an Endonuclease and a DNA Alkylating Agent

During the virus isolation process of samples JP125 and NCP124, the DNA content was analyzed at different steps, as indicated in Table 8, by THRESHOLD™ assay, as specified in section 6 of Example 3. A clearance factor was calculated to evaluate the efficiency of each step in eliminating contaminating DNA, consisting in dividing the DNA amount from the first step by the DNA amount from the following step. A total clearance factor was also calculated to evaluate the overall efficiency of the whole process in eliminating contaminating DNA

TABLE 8
DNA clearance along the virus isolation phase

| | JP125 | | NCP124 | |
|---|---|---|---|---|
| | mg | Clearance | | Clearance |
| Process steps | DNA | factor | mg DNA | factor |
| Harvest | 117 | | 241 | |
| Clarified harvest | 121 | 1 | 35.7 | 7 |
| BPL | − | − | + | + |
| Ultrafiltration | | | | |
| Retentate before | 100 | 1 | | |
| BENZONASE ™ | | | | |
| Retentate after | 3.6 | 28 | 1.9 | 19 |
| BENZONASE ™ | | | | |
| BPL | + | + | + | + |
| Ultracentrifugation 1 | 0.58 | 5 | 0.0006 | 322 |
| Ultracentrifugation 2 | 0.006 | 76 | 0.0059 | 0.1 |
| Anion Exchange | 0.002 | 3 | | |
| membrane | | | | |
| Total clearance factor | | 58500 | | 41000 |

Results—Conclusion

Several process steps contribute to the DNA clearance, but the contribution of BENZONASE™ and ultracentrifugation steps are clearly significant. It can be indirectly concluded from this experiment that BENZONASE™ results in DNA degradation, as a dramatic decrease of the DNA amount was observed (JP125 sample) after the retentate was subjected to a BENZONASE™ degradation. As no further purification step was implemented after degradation and before measuring the DNA amount, the observed decrease reflects DNA degradation, as the THRESHOLD™ assay only guarantees an optimal detection of DNA fragments whose length is greater than 800 base pairs (Briggs and Panfili, 1991, Anal. Chem. 63(9): 850-859). DNA fragments whose length is less than 800 base pairs, depending on their size, may not be detected as efficiently by this assay.

With a total clearance factor of 58500 and 41000, the method according to the invention allows to reduce residual host cell DNA by more than 99.99%, as compared to the amount initially present in the viral harvest obtained by collecting the cell culture supernatant of infected cells.

### Example 9

### HA Yield after Multiple DNA Degradation Steps Performed with an Endonuclease and a DNA Alkylating Agent

HA yield has been calculated by the SRD method as described below at the main steps of the virus isolation process of samples NCP124 and JP125, as indicated in Table 9. The obtained values are all to be compared with the values obtained at the preceding step.

TABLE 9
HA yield

| | SRD HA yield (%) | | | |
|---|---|---|---|---|
| | | Ultra- | Ultra- | Ultra- |
| Sample | Clarification | filtration | centrifugation 1 | centrifugation 2 |
| NCP124 | 93 | 78 | 54 | 102 |
| JP125 | 90 | 63 | 42 | 83 |

SRD Method Used to Measure HA Content

Glass plates (12.4-10 cm) are coated with an agarose gel containing a concentration of anti-influenza HA serum that is recommended by NIBSC. After the gel has set, 72 sample wells (3 mm diameter) are punched into the agarose. 10 μl of appropriate dilutions of the reference and the sample are loaded in the wells. The plates are incubated for 24 hours at room temperature (20 to 25° C.) in a moist chamber. After that, the plates are soaked overnight with NaCl solution and washed briefly in distilled water. The gel is then pressed and dried. When completely dry, the plates are stained on Coomassie Brilliant Blue solution for 10 minutes and destained twice in a mixture of methanol and acetic acid until clearly defined stained zones become visible. After drying the plates, the diameter of the stained zones surrounding antigen wells is measured in two directions at right angles. Alternatively equipment to measure the surface can be used. Dose-response curves of antigen dilutions against the surface are constructed and the results are calculated according to standard slope-ratio assay methods (Finney, D. J. (1952). Statistical Methods in Biological Assay. London: Griffin, Quoted in: Wood, J M, et al (1977). J. Biol. Standard. 5, 237-247)

### Example 10

### HA Purity Evolution During the Virus Isolation Process Comprising Multiple DNA Degradation Steps Performed with an Endonuclease and with a DNA Alkylating Agent

At the mains steps of the virus isolation process of samples NCP124 and JP125, as indicated in Table 10, the SRD/Protein ratio has been calculated, representing the specific amount of HA that is detectable by the SRD method over the total proteins. The concentration of total proteins is measured by the classical Lowry method.

Results are presented in Table 10.

A “purification factor” has also been calculated so as to represent the evolution of the purification during the virus isolation process. Results are presented in Table 11.

TABLE 10
HA purity

| | SRD/Protein (%) | | | | |
|---|---|---|---|---|---|
| | | | Ultra- | | |
| | | | Centri- | Ultra- | |
| | | Ultra- | fugation | Centrifugation | Purified |
| Sample | Clarification | filtration | 1 | 2 | bulk |

| | | | | | |
|---|---|---|---|---|---|
| NCP124 | 2.9 | 8.1 | 17 | 30 | 28 |
| JP125 | 5.4 | 11.3 | 20.4 | 30 | 35 |

TABLE 11
Purification factor

| | Purification factor | | |
|---|---|---|---|
| Sample | UF*/Clarif | UCF**1/Ultra | UCF**2/UCF**1 |
| NCP124 | 2.8 | 2.1 | 1.8 |
| JP125 | 2.1 | 1.8 | 1.5 |

*UF: Ultrafiltration
**UCF: Ultracentifugation

### Example 11

### Immunogenicity of MDCK-Produced Influenza Virus Antigen Subjected to Multiple DNA Degradation Steps Performed with an Endonuclease and a DNA Alkylating Agent

Immunogenicity of the sample NCP124 was assessed (i) in a naive mouse model aimed at reproducing the immunological state of seronegative young children who have never been exposed to influenza antigens and (ii) in a primed mouse model aimed at reproducing more closely the immunological state of seropositive elderly humans who have previously encountered influenza antigens.

Immunogenicity was evaluated by analyzing the hemagglutination inhibition titers and the in vitro neutralization titers. In all experiments, immunogenicity was compared to immunogenicity of egg-derived whole inactivated influenza virus.

### 11.1 Hemagglutination Inhibition (HI) Test

The principle of this HI test is based on the ability of specific anti-Influenza antibodies to inhibit hemagglutination of chicken red blood cells (RBC) by Influenza virus hemagglutinin complex (HA). Sera (50 μl) are treated with 200 μl RDE (receptor destroying enzyme) for 16 hours at 37° C. The reaction is stopped with 150 μl 2.5% Na citrate and the sera are inactivated at 56° C. for 30 minutes. A dilution 1:10 is prepared by adding 100 μl PBS. Then a 2-fold dilution series is prepared in 96 well plates (V-bottom) by diluting 25 μl serum (1:10) with 25 μl PBS. 25 μl of the reference antigens are added to each well at a concentration of 4 hemagglutinating units per 25 μl. Antigen and antiserum dilution are mixed using a microtiter plate shaker and incubated for 60 minutes at room temperature. 50 μl of chicken red blood cells (RBC) (0.5%) are then added and the RBCs are allowed to sediment for 1 hour at room temperature. The HI titer corresponds to the inverse of the last serum dilution that completely inhibits the virus-induced hemagglutination.

### 11.2 In Vitro Neutralization Assay.

Serial 2-fold dilutions of heat-inactivated sera (50 μl) were incubated with 50 μl of each reference antigen in 96 well plates for 1 h30 at room temperature. 100 μl of MDCK cells (2.4×10⁵ cells/ml) were then added to the wells and incubated at 35° C. for 7 days. After incubation, virus-induced cytopathic effect was visually scored in each well under the microscope. The neutralizing titers were expressed as the inverse of the highest dilution of serum that completely inhibits cytopathic effect.

### 11.3 Naive Mouse Model

Mice were immunized by the intramuscular route on day 0 with cell-derived influenza virus antigen (sample NCP24; 1.5 μg HA content) or egg-derived influenza virus antigen (1.5 μg HA content). On day 28, mice were given an identical second immunization.

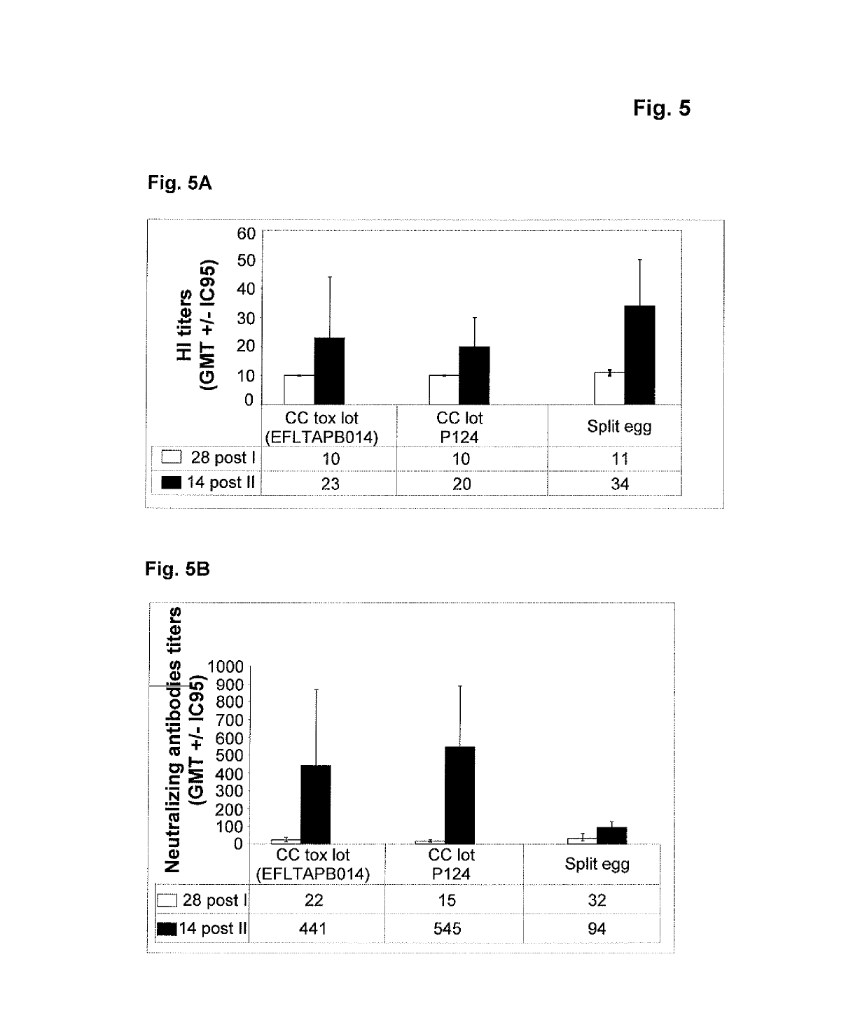

The HI antibody response (determined according to the method of section 11.1) was measured on sera sample collected 28 days after the first immunization and 14 days after the second immunization. Samples were tested for their ability to inhibit hemagglutination of A/New Caledonia/20/99 H1N1 strain. Results are shown in FIG. 5A.

A similar response was obtained, as no statistical difference was observed between the cell culture-based Influenza virus antigen and the egg-derived Influenza virus antigen.

The neutralizing antibodies response (determined according to the method of section 11.2) was measured on sera sample collected 28 days after the first immunization and 14 days after the second one. Samples were tested for their neutralizing activity against A/New Caledonia/20/99 H1N1 strain. Results are shown in FIG. 5B.

A higher neutralizing titer was observed for the cell culture-based Influenza virus antigen, as compared to the egg-derived Influenza virus antigen.

Therefore, the addition of BENZONASE™ and BPL had no negative impact on immunogenicity of the cell-derived Influenza virus antigen, and in this experiment, led to a higher neutralizing response antibody.

### 11.4 Primed Mouse Model

Mice were primed by the intranasal route on day 0 with 5 μg of whole inactivated Influenza virus (A/New Caleddonia/20/99 H1N1). On day 28, the animals were vaccinated by the intranasal route with cell-derived influenza virus antigen (sample NCP24; 1.5 μg HA content) or egg-derived influenza virus antigen (1.5 μg HA content).

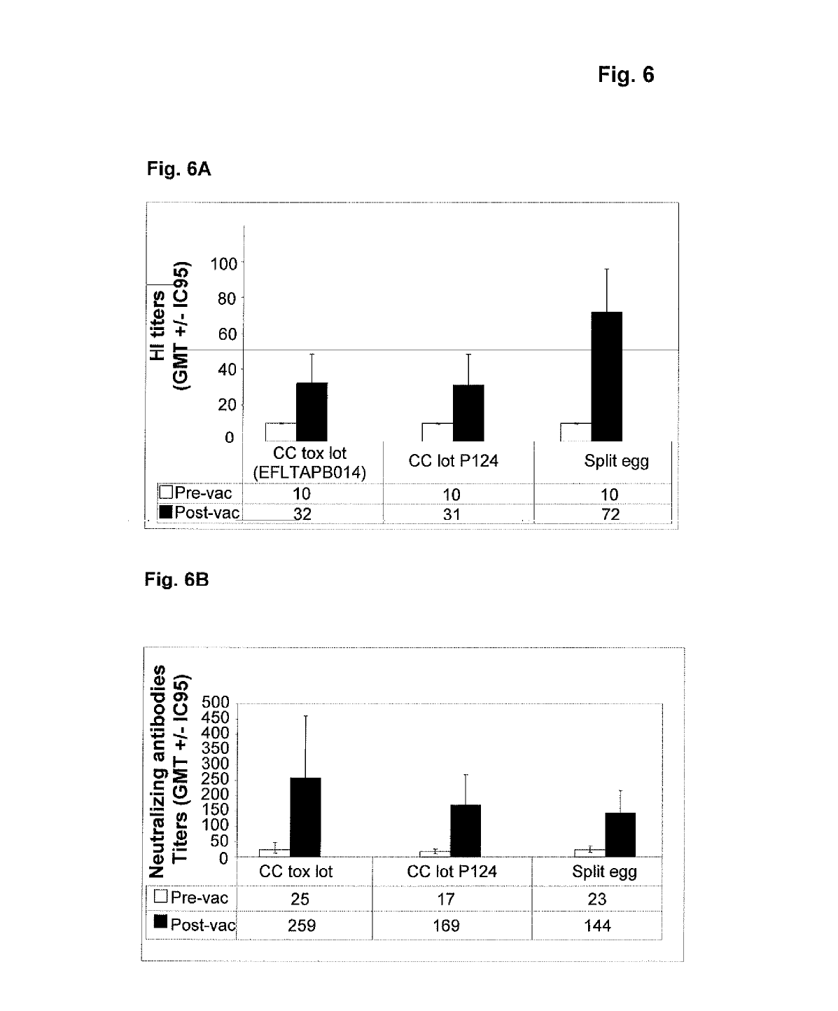

The HI antibody response (determined according to the method of section 11.1) was measured on sera sample collected 28 days after priming and 14 days after vaccination. Samples were tested for their ability to inhibit hemagglutination of A/New Caledonia/20/99 H1N1 strain. Results are shown in FIG. 6A.

A higher HA antibody response was observed for the egg-derived Influenza virus antigen, as compared to the cell culture-based Influenza virus antigen.

The neutralizing antibodies response (determined according to the method of section 11.2) was measured on sera sample collected 14 days after the vaccination and tested for their neutralizing activity against A/New Caledonia/20/99 H1N1 strain. Results are shown in FIG. 6B.

A similar neutralizing titer was observed for the cell culture-based Influenza virus antigen and the egg-derived Influenza virus antigen after priming with whole egg virus.

### Example 12

### Implementation of a Detergent Storage Step after the Splitting Sucrose Gradient Ultracentrifugation

Virus multiplication in MDCK cells, as well as virus harvest and virus isolation were mainly performed as described in Example 2, with the following modifications:
During the step c) of the virus isolation phase, BENZONASE™ was added to the retentate at a final concentration of 135 units/ml.
During the step e) of the virus isolation phase, the second sucrose gradient ultracentrifugation was performed in the presence of 1.5% Triton X-100 (NCP111) or 1% Triton X-100+0.5% SLS (NYP121).

After draining the ultracentrifuge rotor from step d), the HA containing fractions were gathered.

The resulting pool from the sample NCP111 was assayed for the total protein content and diluted twice with PBS—Triton X-100 0.5%—vitamin E succinate 0.1 mM pH 7.4 buffer, filtrated at 20 ml/min onto a 0.22 μm membrane in sterile conditions and further diluted with the same buffer to reach a final protein concentration of 250 μg/ml. The Triton X-100-diluted pool was then left incubated for 72 hours at room temperature or not.

A control was realized by incubating a pool diluted to 250 μg/ml without Triton X-100 addition for 72 hours at a temperature ranging from 2 to 8° C.

The resulting pool from the sample NYP121 was assayed for the total protein content. Aliquots were diluted with a PBS—alpha-tocopheryl hydrogen succinate 0.1 mM pH 7.4 buffer supplemented with Triton X-100 at concentrations ranging from 0 to 0.5%, as indicated in Table 13, to reach a final protein concentration of 250 μg/ml and filtrated onto a 0.22 μm membrane. The Triton X-100-diluted pool was then left incubated for 72 hours at room temperature

The effect of such a storage in the presence of Triton x-100 on the infectivity of the samples NCP111 and NYP121 was analysed by calculating the TCID50/ml (Tissue Culture Infectious dose) which represents the amount of virus able capable of infecting 50% of the cells.

Results are presented in Tables 12 and 13.

TABLE 12
Virus infectivity

| | TCID 50/ml | |
|---|---|---|
| NCP111 - Sample conditions | (log10) | log tot. |
| No storage - No Triton X-100 | 2.88 | 5.48 |
| Storage for 72 h at 4° C. - No Triton X-100 | 1.90 | 4.19 |
| No storage - 0.5% Triton X-100 | 1.80 | 4.40 |
| Storage for 72 h at RT - 0.5% Triton X-100 | <0.8 | — |

Results

After 72 hours at 4° C., residual virus was detected if the ultracentrifuge pool was diluted with a buffer containing no Triton X-100, as a TCID50/ml titer of 1.90 was observed. A similar observation was made when Triton X-100 was added, but no storage was implemented, as a TCID50/ml titer of 1.8 was observed. When incubated at room temperature in a buffer containing 0.5% Triton X-100, the observed TCID50/ml titer was <0.8, i.e. below the limit of detection of the assay, indicating that the residual virus infectivity was eliminated.

TABLE 13
virus infectivity

| NYP121 - Sample conditions | TCID 50/ml | |
|---|---|---|
| 72 h storage at room temperature with: | (log10) | log tot. |
| 0% Triton X-100 | 2.11 | 2.81 |
| 0.1% Triton X-100 | 0.93 | 1.63 |
| 0.3% Triton X-100 | <0.8 | <1.5 |
| 0.5% Triton X-100 | 1.8 | <3.46 |

Results

After 72 hours at room temperature, residual virus is detected if the ultracentrifuge pool was diluted with a buffer containing no or 0.1% Triton X-100. In the case of a dilution with a buffer containing 0.3% Triton X-100, a TCID 50/ml titer<0.8 was observed. With a 0.5% triton X-100, a buffer toxicity was observed and a titer of less than 1.8 could be deduced from the experiment.

Conclusions

Therefore, the split virus pool collected from sucrose gradient comprising either Triton (NCP111) or a mixture of Triton and SLS (NYP121) as a splitting detergent, contains a significant residual infectivity. Incubation of the split virus, after splitting occurred, for up to 72 hours at 4° C. (NCP111) or at room temperature (NYP121), does not allow eliminating the residual infectivity, despite the presence of a low residual concentration of the splitting detergent. This problem can be solved by storing the virus preparation for a few days in the presence of detergent, for further inactivating the infectious virus still present after the splitting step.

Indeed, adding Triton X-100 to the dilution buffer of the split virus pool allows reducing the residual infectivity, yet a minimum concentration of 0.3% is required to reduce the infectivity to undetectable levels.

### Example 13

### Separation of MDCK Proteins from the Viral Proteins by a Gel Filtration Chromatography in the Presence of a Zwitterionic Detergent

Virus multiplication in MDCK cells, as well as virus harvesting and virus isolation were mainly performed as described in Example 2, with the following modifications:
After the ultrafiltration step i), a final concentration of 0.1% empigen is added to the retentate removed from the ultrafiltration system. The mixture is stirred for 15 minutes and then loaded onto a Sephacryl S200 column (GE Healthcare), equilibrated in a PBS-0.1% SLS buffer. The separation is run at a linear flow rate of 30 cm/h at room temperature. The fractions are collected and analyzed by SDS-PAGE and Western-blot using anti-HA and anti-MDCK antibodies. The viral proteins-containing fractions are pooled while the other fractions containing mainly MDCK proteins are discarded.

The analysis of proteins contaminants was also analyzed by THRESHOLD™ assay, while the detergent added to the retentate and to the equilibration buffer was SLS at a final concentration of 0.1%. Results obtained with the sample NCP124, before and after the step of gel filtration, is presented in Table 12.

TABLE 14
Host Cell Protein content

NCP124

| | | |
|---|---|---|
| Gel Filtration | − | + |
| HCP* | 10 | 4 |
| THRESHOLD ™ % | | |

*HCP is for Host Cell Proteins

Results and Conclusion

The implementation of a gel filtration step in the presence of a detergent has been introduced in order to further decrease the residual MDCK protein content. The addition of 0.1% empigen in the sample (retentate) and the presence of SLS in the equilibration buffer allows a better separation of the contaminants, as revealed by Western blot using anti-MDCK antibodies, when compared to the same protocol implemented in the total absence of detergent. The same separation effect is observed if 0.1% empigen is replaced by 0.1% deoxycholate or 0.1% sarcosine.

The observation of a decrease of the residual MDCK proteins after implementing a step of gel filtration in the presence of a detergent is confirmed by the results obtained by THRESHOLD™ assay, where the amount is reduced by more than 50%, as compared with no gel filtration.

### Example 14

### Combination of a Sucrose Gradient Ultracentrifugation Step and Chromatography Steps for Virus Isolation

Virus multiplication in MDCK cells, as well as virus harvesting and virus isolation were mainly performed as described in Example 2, with the following two sets of modifications:

14.1
The second sucrose gradient ultracentrifugation splitting step e) is missing.
The splitting of the virus is performed in batch.
A hydroxyapatite chromatography is implemented

At room temperature, the first sucrose gradient pool is diluted under stirring with PBS pH 7.4-Triton X-100 to reach a final detergent concentration of 0.5%. Mixing is performed for 2 minutes and is followed by a 15 minutes rest. This operation is repeated once. Acropak 500 (Pall, ref. 12991) 0.8+0.2 μm filter module is used to clarify the splitted material. The membrane is rinsed with 50 ml PBS pH7.4—Triton X-100 0.5% to optimize the protein recovery. The filtrated material is then diluted with 1 M PO4, pH 7.0, Nacl 0.2 M before the chromatography step. A 40 ml hydroxyapatite column XK16/20 is equilibrated in 50 mM PO4, pH 7.0, NaCl 100 mM buffer. The feed is injected at a linear velocity of 200 cm/h. Viral proteins are collected within the flow through. After a 0.22 μm filtration, formaldehyde inactivation is performed according to step f) of the Example 1, and the process is carried on with the remaining steps g) to j).

14.2
The first sucrose gradient ultracentrifugation of step d) is missing.
A hydroxyapatite or Sephacryl HR200 chromatography is implemented.
   14.2.1

The gradient pool collected after the splitting ultracentrifugation of step e) is diluted, 0.22 μm filtrated and inactivated with formaldehyde according to step f) of the Example 1. After performing the steps g) and h) of the Example 1, a Sephacryl HR200 gel permeation chromatography was implemented. The feed, after addition of 0.1% sarcosyl, is loaded on a 1750 ml XK50 column equilibrated with PBS pH 7.4-0.1% sarcosyl at a linear flow rate of 30 cm/h. The fractions containing the viral proteins are pooled, concentrated with a 30 kDa ultrafiltration membrane and filtrated on a 0.22 μm filter.

14.2.2

Alternatively, the gradient pool after the splitting ultracentrifugation of step e) is diluted with 400 mM NaPO4 pH 7.0-0.1 M NaCl before injection on a 70 ml hydroxyapatite XK16 column equilibrated in 10 mM NaPO4 pH 7.0-0.1 M NaCl at a linear velocity of 100 cm/h. A 0.22 μm filtration of the chromatography ends the process.

Results

14.1

The batch splitting followed by hydroxyapatite leads to a HA purification yield of 31% with a host cell protein contaminant content by THRESHOLD™ of 15% and a SRD/prot ratio of 29%. Dna content is below the specifications (1.56 ng/45 μg HA). Stability data after 28 days at 30° X show 100% HA recovery. No HA (step yield 100% by SRD) nor toher viral proteins (SDS-PAGE silver staining) loss can be observed after hydroxyapatite chromatography, but a few contaminants are removed. Therefore, this process offers the advantage of being simplified over a two-ultracentrifugation steps process. The purified antigen quality (residual host cell proteins and DNA) is comparable with the one obtained with the two-sucrose gradient ultracentrifugation steps process. However, the yield of purified HA is increased by about 2-fold.

14.2.1

The implementation of a Sephacryl HR200 gel permeation chromatography after a splitting ultracentrifugation step leads to a HA purification yield of 18%, with a host cell protein contaminant content by THRESHOLD™ of 4% and a SRD/prot ratio of 32%. DNA content is below the specifications (1.87 ng/45 μg HA). The yield is comparable to that of the two-sucrose gradient ultracentrifugation steps process. However, the level of residual host cell protein contaminant is significantly reduced.

14.2.2

The implementation of a hydroxyapatite chromatography after a splitting sucrose gradient ultracentrifugation step leads to a HA purification yield of 25%, with a host cell protein contaminant content by THRESHOLD™ of 23% and a SRD/prot ratio of 22%. This alternative allows for a better yield than the two-sucrose gradient ultracentrifugation steps process. However, the purity (SRD/pot ration and host cell protein residual) is somewhat lower.

Conclusions

Alternatively to the process described in Examples 1 and 2, based on two ultracentrifugation steps performed successively, it is feasible to implement only one ultracentrifugation step, in combination with a chromatography step. The splitting can be performed either in the sucrose gradient, or in batch, after virus concentration and isolation by ultracentrifugation. This alternative allows for a better antigen yield along the isolation process.

### Example 15

### Production of Influenza Virus in EB66™ Cells in the Presence of Multiple DNA Degradation Steps Performed with an Endonuclease and an Alkylating Agent

In order to demonstrate that the method according to the invention can be used in another cell type and is at least as efficient for degrading DNA and eliminating it as it is when implemented in MDCK cells, Influenza virus has been produced in EB66™ (WO 2008/129058) and an experiment based on combining multiple DNA degradation steps performed with BENZONASE™ and BPL has been conducted, in part based on the experiment described in Example 2.

Briefly, EB66™ cells were grown in suspension in a batch mode. They were infected with H5N1 and the virus was harvested by collecting the cell culture medium a few days later. BENZONASE™ was added to the cell culture medium after the virus was inoculated into the cells. The viral harvest was clarified and the clarified harvest treated with BPL. After concentration of the BPL-treated harvest by ultrafiltration, BENZONASE™ was added to the retentate removed from the ultrafiltration system. The virus was then further purified, in particular, through two successive sucrose gradient ultracentrifugations. The sucrose gradient of the second one contained Triton X-100 so as to also split the virus. Residual host cell DNA was then eliminated from the virus preparation by filtering the preparation on an anion exchange membrane. The resulting virus preparation was referred to as the purified bulk

Characterization of Residual EB66™ Cells DNA in the Purified Bulk

The concentration of total DNA amount, in the purified bulk, was measured by the THRESHOLD™ assay, as described in section 7 of Example 3. In order to determine the size distribution of the residual host cell DNA left in the purified bulk, the concentration of DNA fragments of 99 bp, 185 bp and 280 bp was analysed by quantitative PCR with respective primers, as described in section 5 of Example 3. In parallel, the HA concentration, in the purified bulk, was determined via SRD assay (see Example 9). Then, the DNA values were normalized against 45 μg HA, the expected regular dose of a trivalent vaccine containing 15 μg HA of each strain. The results obtained are presented in Table 15 and Table 16.

TABLE 15
DNA amount in purified bulk

| | THRESHOLD ™ | THRESHOLD ™ |
|---|---|---|
| HA (μg/ml) | (ng/ml) | (pg/45 μg HA) |
| 144 | 0.12 | 36.9 |

TABLE 16
DNA size distribution in purified bulk

| | Q-PCR ng/ml | | | Q-PCR (pg/45 μg HA) | | |
|---|---|---|---|---|---|---|
| HA (μg/ml) | 99 bp | 185 bp | 280 bp | 99 bp | 185 bp | 280 bp |
| 144 | 0.02 | 0.01 | 0.01 | 6.228 | 6.249 | 3.129 |

Results—Conclusion

Implementing multiple DNA degradation steps, according to the method of the invention, in EB66™ cells provide purified bulks of virus comprising very low amount of residual host cell DNA. The total DNA amount, as measured by THRESHOLD™ assay, i.e. irrespective of its size, and as normalized against 45 μg of HA is as low as 36.9 pg. Compared to the results obtained in Example 6 relating to purified bulks originating from MDCK cells and displaying residual host cell DNA ranging from 0.2 to 0.4 ng/45 pg HA, the value obtained in EB66™ cells is at least 5 times less and up to 10 times less.

The Q-PCR results targeting DNA sequences of 99 bp, 185 bp and 280 bp also provided very good results, as only 3.1 pg of DNA/45 μg HA with respect to the fragment of 280 bp was detected in the purified bulk, as well as only 6.2 pg of DNA/45 μg HA with respect to fragments of 185 bp and 280 bp was detected in the purified bulk. These results are within the same range as the DNA amount observed in purified bulks originating from MDCK cells (Example 6), indicating that the method of the invention does not only provide low DNA results when a virus is produced on MDCK cells, but also when a virus is produced on EB66™ cells. And the obtained results are far below the specification of 10 ng DNA/vaccine dose by the Health Authorities.

1. A method for producing a virus or a viral antigen thereof in cell culture comprising the steps of:
(a) providing a population of cells cultured in a cell culture medium,
(b) inoculating the population of cells with a virus,
(c) culturing the population of cells so as to allow the virus to replicate,
(d) collecting the produced virus thereby providing a viral harvest, and
(e) isolating the virus,
the method comprising at least one step of host cell nucleic acids degradation with an endonuclease and at least one step of host cell nucleic acids degradation with a DNA alkylating agent.

2. The method according to claim 1, wherein at least one step of host cell nucleic acids degradation is implemented before step (d).

3. The method according to claim 2, wherein the at least one step of host cell nucleic acids degradation is performed with the endonuclease.

4. The method according to claim 3, wherein the endonuclease is added to the cells in culture after their inoculation with the virus.

5. The method according to claim 1, wherein the population of cells is cultured in a bioreactor.

6. The method according to claim 5, wherein the endonuclease is added to the bioreactor.

7. The method according to claim 1, wherein the culture medium is provided by perfusion.

8. The method according to claim 7, wherein the endonuclease is added to the perfusion medium.

9. The method according to claim 1, wherein the endonuclease is added to the viral harvest obtained after step (d).

10. The method according to any claim 1, wherein at least one step of degradation of the host cell nucleic acids is implemented during the virus isolating step.

11. The method according to any claim 1, wherein the virus isolating step comprises at least one step selected from viral harvest clarification, ultrafiltration/diafiltration, ultracentrifugation and chromatography, or any combination thereof.

12. The method according to claim 11, wherein the virus isolating step comprises at least a combination of: a viral harvest clarification step, an ultrafiltration/diafiltration step thereby producing a retentate and one or two ultracentrifugation steps.

13. The method according to claim 12, wherein at least one ultracentrifugation step is a sucrose gradient ultracentrifugation step.

14. The method according to claim 11, wherein the isolating step comprises at least one step of sucrose gradient ultracentrifugation.

15. The method according to claim 10, wherein the at least one step of degradation of the host cell nucleic acids is performed with the endonuclease.

16. The method according to claim 15, wherein the endonuclease is added to the retentate obtained after ultrafiltration/diafiltration.

17. The method according to claim 10, wherein the at least one step of degradation of the host cell nucleic acids is performed with the DNA alkylating agent.

18. The method according to claim 17, wherein the DNA alkylating agent is added to the clarified viral harvest.

19. The method according to claim 17, wherein the DNA alkylating agent is added to the retentate obtained after ultrafiltration/diafiltration.

20. The method according to claim 1, further comprising a virus splitting step performed in batch.

21. The method according to claim 20, wherein Triton X-100, optionally combined with deoxycholate or sodium lauryl sulfate, is used for the splitting step.

22. The method according to claim 1, wherein the alkylating agent is beta-propiolactone.

23. The method according to claim 1, wherein the cells in culture are selected from the group consisting of mammalian cells and avian cells.

24. The method according to claim 23, wherein the cells are MDCK cells.

25. The method according to claim 23, wherein the cells are avian cells derived from duck embryonic stem cells.

26. The method according to claim 25, wherein the cells are EB66® cells.

27. The method according to claim 1, wherein the virus is influenza virus.

28. The method according to claim 27, wherein the influenza virus is of a pandemic or potentially pandemic strain.

29. The method according to claim 1, further comprising inactivating the virus with one or more inactivating agents.
